(19) United States
(12) Patent Application Publication
(10) Pub. No.: US 2026/0311865 A1
(43) Pub. Date: Oct. 8, 2026

(54) System and Method for Age Deceleration and Preventative Health

(71) Applicant: LifeQ B.V., Amsterdam (NL)

(72) Inventors: Franco Bauer du Preez, Cumming, GA (US); Laurence Richard Olivier, Alpharetta, GA (US)

(21) Appl. No.: 19/482,568

(22) PCT Filed: May 8, 2024
(86) PCT No.: PCT/US2024/028375

§ 371 Date: Nov. 7, 2025

Related U.S. Application Data
(60) Provisional application No. 63/500,711, filed on May 8, 2023.

(51) Int. Cl. G16H 40/67; A61B 5/00; A61B 5/024; A61B 5/11

(52) U.S. Cl. CPC G16H 40/67; A61B 5/02416; A61B 5/02438; A61B 5/1126; A61B 5/681; A61B 5/721; A61B 5/7275

(57) ABSTRACT

The invention harnesses PPG-based wearable device technology to predict Wearable Derived Biological Age Acceleration (WDBAA) by utilizing a feature set known as the wearable-ome. This model is aligned with leading hypotheses in gerontology and is trained against measures of Biological Age Acceleration (BAA). The system offers real-time feedback on BAA, correlating behavioral changes and lifestyle interventions with shifts in BAA, while optimizing intervention strategies to minimize user friction. Additionally, this invention uses periodic Epigenetic Biological Age (EBA) assessments to enhance model accuracy and inform users of changes in key longevity genes such as MTOR, sirtuins, and AMPK, known to influence longevity. An unpublished manuscript substantiates the model's capability to predict epigenetic aging, providing users with actionable insights into physiological and behavioral factors contributing to BAA. The wearable-ome facilitates a detailed understanding of factors accelerating biological aging, enhancing user engagement and intervention effectiveness.

### CROSS-REFERENCE TO RELATED APPLICATIONS

This invention claims priority from U.S. Provisional Patent Application Ser. No. 63/500,711, filed May 8, 2023, which is incorporated by reference in their entirety herein.

### BACKGROUND

Aging is the time-dependent process of cellular and physiological decline of an organism, ultimately, leading to death, which is accompanied by an increased risk for age-related diseases such as cardiovascular and neurodegenerative diseases, and cancers. Life expectancy projections estimate that by the year 2050 at least 25% of the developed world's population will be over the age of 65. The portion of life free from chronic disease, healthspan, is not increasing in the same manner. Measuring the organismal state, biological age, and understanding the molecular and physiological mechanisms, how to track them, and influence the underlying processes is crucial for delaying the onset of chronic disease, and prolonging both healthspan and lifespan.

Efforts have been made to understand the mechanisms of aging, whereby the hall marks have been identified, categorized as: primary hallmarks (the cause of damage), antagonistic hallmarks (the response to damage), and integrative hallmarks (the consequences of the phenotype). These provide a landscape to understand the intricate and complex networks present in aging. A class of epigenetic modifications characterizing aging is DNA methylation (DNAm) of the cytosine residue usually found at cytosine-guanine dinucleotides (CpGs). As aging progresses, these modifications occur where methylation status of specific CpG sites change predictably, differential methylation, and variably, variable methylation.

Epigenetic clocks are designed to determine biological age based on measuring the epigenetic modifications that accompany aging. The first-generation clocks have been trained to predict chronological age, whilst second generation clocks are trained on age-related outcomes. DNAm age has been shown to be predictive of all-cause mortality (ACM), healthspan, and physical function. An additional layer of biological context is revealed when addressing the biological age acceleration, defined as the residual of the linear model of biological age regressed onto chronological age. An increased age acceleration is associated with decreased lung function, cardiovascular diseases, neurodegenerative diseases, cancer, and mental illness. Lifestyle factors, such as exercise, sleep disorder and smoking status are associated with age acceleration. On the molecular level, age acceleration has been associated with key pathways involved in cellular homeostasis, inflammation, cell division, and metabolism, to name a few. Limitations of these clocks include that not all the clocks agree with each other on an individual level, possibly as the clocks are trained to predict different aspects of aging, as previously explained. Additionally, technical variance is introduced on the individual level, whereby repeat measures result in large variance. This has been addressed by retraining the clocks using a principal component analysis (PCA) method, significantly decreasing the variation of repeated testing. On a commercialization level, epigenetic clocks present friction in the invasive and expensive nature of the biomarker, although recent work suggests a reduction in the cost of analysis. Due to the challenges set out above, an accurate, non-invasive, in-expensive, continuous estimate of biological age is lacking.

In the search for advice on how to create a lifestyle that optimizes healthspan, individuals face a wealth of information that ranges across a wide span of veracity. Even experts lack an approach that goes beyond the methodology of creating a systematic review or meta-analysis, where there are established methods for dealing with heterogeneous degrees of evidence seen in scientific studies.

Here are some exemplary categories:

Longitudinal studies: These are studies that follow a group of individuals over an extended period of time, collecting data at multiple points. Longitudinal studies can help establish temporal relationships between exposures and outcomes and are often used to investigate causal relationships.

Randomized controlled trials (RCTs): These are studies that randomly assign participants to different groups, such as a treatment group and a control group, and compare the outcomes between the groups. RCTs are considered the gold standard for investigating causal relationships because they are designed to minimize bias and provide strong evidence for cause-and-effect relationships.

Quasi-experimental studies: These are studies that use a comparison group to investigate the effects of an intervention, but the comparison group is not randomly assigned. Quasi-experimental studies can be useful for investigating causal relationships in situations where RCTs are not feasible or ethical.

Case-control studies: These are studies that compare individuals with a particular outcome, such as a disease, to individuals without the outcome and look for differences in exposure to a particular risk factor. Case-control studies are useful for identifying potential risk factors or associations but are not as strong as RCTs for establishing causality.

Cohort studies: These are studies that follow a group of individuals over a period of time and collect data on various outcomes. Cohort studies can provide valuable information on the natural history of a disease or condition, and can help identify risk factors or predictors of outcomes.

Downsides and upsides of current scientific evidence

By organizing studies into these evidence categories, researchers can assess the quality and strength of the available evidence on a particular topic and draw conclusions or make recommendations based on the collective findings of the studies included in the meta-study or systematic review. Researchers also have to consider the statistical strength of the findings, which varies widely due to factors that include heterogeneous study sizes.

The above demonstrates that it is not a simple task for an individual to even rank evidence available on individual lifestyle interventions for optimizing their healthspan, let alone rank the impact of different combinations of lifestyle interventions using evidence on individual interventions. Much more can be done to provide access to accurate and personalized lifestyle programs for extending human healthspan.

Although longitudinal studies provide an excellent degree of evidence, and while the same can be said for randomized controlled trials, each has specific shortcomings. Longitudinal studies typically lack interventions, since these are hard to sustain over the course of decades and therefore only deliver associations/correlations, but not causal relationships. These studies are also running on a timescale that is impractical for learning which interventions can most effectively extend human healthspan.

Randomized controlled trials are costly, requiring large numbers of participants to produce population level findings that reach significance for a short-term intervention, instead of findings that are statistically significant with regard to intervention in a particular individual in isolation.

### SUMMARY OF THE INVENTION

It is to be understood that this summary is not an extensive overview of the disclosure. This summary is not restrictive, and it is intended to neither identify key or critical elements of the disclosure nor delineate the scope thereof. The sole purpose of this summary is to explain and exemplify certain concepts of the disclosure as an introduction to the following complete and extensive detailed description.

Some of these challenges can be overcome by aiming for more detailed and comprehensive outcome measures versus the simpler clinical end-points that are currently in use, like disease status, all-cause mortality or a small collection of health biomarkers like blood pressure or cholesterol. A concrete example of such a comprehensive outcome measure would be to obtain high throughput biology data (HBD), such as a methylome, proteome or transcriptome data for each participant. This gives an indication of which genes have been activated by a specific lifestyle intervention or drugs. Within these comprehensive outcomes, it is possible to assess the functional state of cellular components (FSCC). Some cellular components are known to causatively and longitudinally extend healthspan through experiments where they have been directly bioengineered to have increased or decreased activity levels, we have termed this subset here as Universal ProLongevity Genes (UPLG). In this manner, some level of longitudinal causal understanding is achieved, albeit on the partial phenotype and not the complete phenotype of a person undergoing an intervention. This level of causality can be seen as intermediate between a randomized control trial (full causality) and an observational study where narrow sets of correlative relationships are discovered. By focusing on cellular components that act as master regulator switches that have a large causative downstream domain (i.e. regulate thousands of downstream components), this partial domain of causality can be greatly broadened compared to a random cellular component. Well known examples of such components include AMPK, mTOR and the sirtuins, each of which have been shown to impact longevity directly and universally across a wide range of animal models in this manner. Transcription factors, such as hypoxia inducible factor, which drives increases in aerobic fitness, directly regulate the transcription of hundreds of genes. It would also be possible to assess the activation of cellular components known to have negative effects.

By having such a detailed view, it becomes more feasible to investigate which interventions would be complementary by activating non-overlapping beneficial cellular components to achieve a positive outcome more effectively when combined versus the opposite scenario, where two interventions might function via the same cellular pathways and potentially provide a less powerful combination. It might also be possible to attenuate negative side effects of specific interventions, by the addition of interventions that are known to specifically attenuate these at the level of cellular component activity identifiable through high throughput biology samples.

Complex biological measures, like epigenome data, which for humans contain ~800,000 methylation states per sample and give information on the regulation of the total set of human genes, can also be regressed onto well understood measures of health, such as for example chronological age to produce a single value measured in years. Such an index, known as a ‘biological age’, has in the literature been shown to be strongly connected to health in a wide range of cases, not only when derived from epigenetic data (Levine et al., 2018), but also when derived from a wide variety of other biomarkers, like ECG parameters, aerobic fitness levels, blood biomarkers (PhenoAge), muscle strength, bone density, etc. By using an index that has already been established to be tightly coupled to the causative mechanism behind the biological aging process, especially in the case of the epigenome, we are essentially creating a study endpoint that provides a broadly informed, but finely graded measure of health (as opposed to low frequency, discrete mortality and morbidity events used traditionally, or low dimensional data like blood biomarkers) that detects the invisible early signatures of aging. This provides immense benefits for studying preventative healthcare interventions, since it can be used 1.) on a short timescale 2.) can produce many outcome measurements continuously on one individual at a time (personalized medicine, causative instead of correlative relationship discovery for both lifestyle factors as measured by wearables as well as interventions, such as pharmacological agents or nutritional supplements).

Another remaining challenge is that lifestyle programs require costly administrators to assess or enforce adherence, before the impact of the intervention on healthspan can be estimated through mathematical models that map quantified behavioral changes (QCB) to quantified changes in health (QCH). Trials can also not proceed in a fashion where they are ongoing and where participants are monitored remotely and where participants are not necessarily synchronized temporally when participating in an ongoing set of trials that test multiple interventions. This challenge can be overcome by providing IOT technology, such as wrist worn wearables and mobile phones, to track adherence to lifestyle choices. Wrist worn wearables can detect actualized QCB in response to an intervention program provided (possibly via mobile phone)—simple examples include exercise duration and intensity as well as sleep amount and timing of going to bed each night. This technology also allows lifestyle intervention studies to be taken from the clinical trial environment to everyday living, as it becomes possible to provide intervention programs in an ongoing manner, while quantifying changes in behavior in line with these recommendations. Since partial compliance to a program can then be quantified, full adherence and enforcement through trial administrators, is no longer required. Sham lifestyle interventions can also be provided to randomized control groups to convert this into the equivalent of a randomized controlled trial. Presently observational studies typically rely on self reported sleep and activity levels, which are highly inaccurate compared to automated monitoring by wearable IOT technology.

Another remaining challenge is that insights based on the study designs described earlier, are generated at a population level, rather than at an individual level. With the description provided earlier, IOT based devices that monitor compliance in combination with high throughput biology samples that are taken periodically to assess the functional state of cellular components, provide a mechanism for discovering causal mechanisms and to quantify the strength of different known causative mechanisms at the level of individual participants. For example, fasting results in body wide changes in gene regulation (which include changes to epigenetic gene regulation that can be measured in the lab). The upregulation of AMPK transcription or the epigenetic modification via methylation that controls its transcription, can be measured periodically through white blood cells using blood samples and compared to interventions in diet, sleep and fasting using an IOT system that tracks compliance via wearable devices as well as smartphones and nearable devices for keeping track of diet, medication or supplement intervention. Individual variability in how strongly AMPK becomes activated by a fixed intervention, e.g. 16 hour fasting, will vary across individuals and this information can be aggregated through transcriptomic and methylomic data gathered over successive interventions measured by IOT and taking periodic measurements of cellular components, in order to provide a personalized fasting window that achieves an intended level of AMPK activation and healthspan extension. Endpoints like AMPk regulation, with a known causative rather than correlative relationship to health, are expected to reduce the influence of confounding variables very significantly when studying health.

Beyond the use of IOT, such as wearable devices, to track compliance to lifestyle intervention programs in the proposed invention, these devices can also measure continuously several physiological processes that are indicative of increased or decreased healthspan. For example, heart rate variability (HRV), Pulse waveform (PWF) and resting heart rate (RHR) each contain information on participant health. These health measures, alongside several others, such as wearable derived hypnograms, can be used to get an integrated view of the higher level functional state of an individual. Functional measures of health that can be estimated from wearables, with VO2max also being a good example, provide a second layer, beyond molecular components, for assessing the integrated outcome of an intervention in an IOT environment. Such integrated assessments can be used to confirm whether the partial causal information derived from molecular changes are producing higher level functional QCH, such as increased VO2max or increased cardiovascular health, bringing successful preventative health intervention into even sharper focus.

Changes in both molecular components measured through high throughput biology samples, as well as physiology, as measured through IOT technology (QCH), can be combined into single indices, called biological ages. Molecular clocks for measuring not chronological, but instead biological age, such as the Horvath clock are known in the scientific literature and can be used to determine whether, from an epigenetic point of view, a participant is accelerating or decelerating their overall biological age. The same concept can be extended to wearable biomarkers that measure human physiology. By considering the impact of combinations of interventions on an individual, it becomes possible to estimate and optimize their joint long term effectiveness for a particular individual.

In an aspect, the disclosure is directed to a system that includes wearable and other IOT technologies for monitoring human behavior and physiology, alongside periodic measurements via high throughput biology of changes in cellular component activity in response to interventions that are trialed under free-living conditions to extend human health span. Knowledge about the long term causative impact of master regulator cellular components, such as AMPK, mTOR and the sirtuins, is used to estimate partial long term causal impact on healthspan of interventions, which are trialed dynamically through the ability of IOT and wearable technology to measure trial compliance in the field. The system takes the process of discovering causal interventions from the clinical trial environment, to the consumer environment, to dramatically scale up the rate at which effective interventions are learned, where interventions include, but are not limited to dietary, supplementation, pharmacological, sleep and exercise.

### DESCRIPTION OF THE FIGURES

The features and components of the following figures are illustrated to emphasize the general principles of the present disclosure. Corresponding features and components throughout the figures can be designated by matching reference characters for the sake of consistency and clarity.

The patent or application file contains at least one drawing executed in color. Copies of this patent or patent application publication with color drawing(s) will be provided by the Office upon request and payment of the necessary fee.

FIG. 1 illustrates a schematic representation of a system according to an aspect of the present invention.

FIG. 2 illustrates a schematic representation of methods and communications performed by the system of FIG. 1 according to an aspect of the present invention.

FIG. 3 illustrates a schematic representation of methods and communications performed by the system of FIG. 1 according to an aspect of the present invention.

FIG. 4 illustrates a clustered heatmap of behavioural, circadian, and physiological features according to an aspect of the present invention. Behavioural and circadian features are plotted as rows, and physiological features as columns. Each cell represents the Pearson r value of the correlations on a sliding colour scale from negative (blue) to positive (red). Significance (p<0.05) is illustrated by a * next to the correlation value in the cell.

FIGS. 5A-5D Illustrate model performance and feature analysis of aspects of the present invention. FIG. 5A is a scatter plot of actual and predicted age acceleration for the training (R2=0.49 [Cls 0.29-0.60]) and testing (R2=0.37) dataset. FIG. 5B illustrates a distribution of actual (blue) and predicted (red) age acceleration over the entire population. FIG. 5C provides a SHAP summary plot giving an overview of feature importance and impact on model predictions. Each point on the plot represents a SHAP value for feature and instance. The position on the y-axis is determined by the feature and on the x-axis by the SHAP value. The colour represents the value of the feature from low (blue) to high (red). Density of datapoints is represented by violin width. FIG. 5D is a representative example of the individual contribution of model features predicting age acceleration for an individual who has a negative age acceleration (−4.5 years) value. Blue bars are representative of the feature reducing age acceleration, and red bars are representative of features increasing age acceleration predictions.

FIGS. 6A-6B illustrate aspects of the WGNA according to an aspect of the present invention. FIG. 6A illustrates a bar plot showing WGCNA module sizes. FIG. 6B illustrates a clustered heatmap of predicted and actual age acceleration, displayed as rows, correlating with the WGCNA MEs, displayed as columns. The heatmap is clustered by columns. Each cell represents the Pearson r value of the correlation on a sliding scale from negative (blue) to positive (red). Significance (p<0.05) is illustrated by a* next to the correlation value in the cell.

FIG. 7 illustrates common enrichment in WGCNA modules and wearable-derived hub sites according to an aspect of the present invention.

FIGS. 8A-8D illustrates pathways of interest according to an aspect of the present invention. The genes present in the pathways are coloured in red indicate annotated genes found in both the wearable derived hub genes and a WGCNA module. FIG. 8A illustrates the longevity regulating pathway. FIG. 8B illustrates the lipolysis in adipocytes pathway. FIG. 8C illustrates the Pl3k-Akt signalling pathway. FIG. 8D illustrates the MAPK signalling pathway.

FIG. 9 lists wearable feature descriptions according to an aspect of the present invention.

FIGS. 10A-10H display wearable feature distributions according to an aspect of the present invention.

FIGS. 11A-11F illustrate sleep PCA according to an aspect of the present invention.

FIG. 12 illustrates a clustered heat map of features vs age correlations according to an aspect of the present invention.

FIG. 13A-13D illustrate training and testing sample splits according to an aspect of the present invention.

FIG. 14 illustrates SGCNA model performance according to an aspect of the present invention.

FIG. 15 illustrates R squared values for the Im predicting each CpG site methylation level.

FIG. 16 lists enriched pathways using wearable features R squared cut off from the 5th percentile of PCPhenoAgeResid R2.

FIG. 17 displays pathway enrichment according to an aspect of the present invention.

FIG. 18 is a graphical representation of Subendocardial Viability Ratio.

FIG. 19 is a graphical representation of the estimate through pulse height.

### DETAILED DESCRIPTION OF THE INVENTION

It should be appreciated that this disclosure is not limited to the devices, systems, components, and methods described herein. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any devices, systems, components, and methods similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or example language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The present disclosure can be understood more readily by reference to the following detailed description, examples, drawings, and claims, and their previous and following description. However, before the present devices, systems, components, and methods are disclosed and described, it is to be understood that this disclosure is not limited to the specific devices, systems, components, and methods disclosed unless otherwise specified, as such can, of course, vary. It is also to be understood that the terminology used herein is for the purpose of describing particular aspects only and is not intended to be limiting.

### Definitions/Acronyms

Biological Age Acceleration (BAA)—Biological age acceleration refers to the phenomenon where an individual's biological age, as determined by various biological markers such as DNA methylation patterns, differs from their chronological age—their age in years since birth. When biological age exceeds chronological age, it indicates accelerated aging, suggesting that the body may be experiencing age-related changes at a faster rate than expected based on calendar age alone.

Epigenetic Biological Age (EBA)

Epigenetic Biological Age Acceleration (EBAA)

Wearable Derived Biological Age Acceleration (WDBAA)

Wearable Derived Biological Age (WDBA)

Quantified changes in health (QCH)—Health in this context refers to objective measures obtained through high-throughput biology and continuous monitoring of physiology via IoT devices, such as wearable or smartphone-based sensors. These measures provide detailed biological data related to health, surpassing the granularity of traditional clinical endpoints like morbidity or mortality events often used in retrospective cohort studies within lifestyle medicine.

Quantified changes in health (QCB)—Behavior in this context refers to objectively measured actions, captured through continuous monitoring of individuals using IoT devices that track movement and physiology. These measurements include parameters such as movement cadence, posture, and activity level, among others. This approach contrasts with the self-reported activity levels typically found in retrospective cohort studies within lifestyle medicine.

High-throughput Biology Data (HBD)—Examples include the methylome (as a subset of the epigenome), transcriptome or proteome for a participant for a tissue like whole blood, but can be other tissue. This gives an indication of which genes have been activated by a specific lifestyle intervention or drugs.

Comprehensive outcome measure (COM)—High throughput biology samples, such as a methylome or transcriptome data.

Functional state of cellular components (FSCC)—A measure relating to the activity level of a cellular component, for example, the frequency of mRNA transcripts, the concentration or phosphorylation state of a particular protein or the methylation/demethylation of DNA elements that influence the expression of a particular gene.

Functional measures of health (FMH)—High level performance state of the body, such as VO2max or epigenetically derived biological age, such as PCPhenoAge (Levine et al., 2018)

Universal ProLongevity Gene (UPLG)—Some cellular components are known to causatively and longitudinally extend healthspan through experiments where they have been directly bioengineered in isolation to have increased or decreased activity levels.

Biological Age Fit (BAF)—The method by which machine learning models, that take HBD as input, are either fitted directly through supervised machine learning or indirectly through generative AI approaches that create latent features that are selected based on their ability to predict the following: An index of health compiled from consideration of chronological age+any health biomarkers relevant to longevity, such as blood biomarkers, mortality, morbidity, VO2max. See for example PCPhenoAge (Levine et al., 2018). In some cases, chronological age is used by itself, without any additional features, see for example Horvath (2013).

Behavior-Physiology Temporal Causal Analysis (BPTCA)—The combined analysis of physiological information (involuntary biomarkers) and behavioral information (voluntary behavior biomarkers such as exercise, activity, sedentary behavior and sleep timing, but also including annotations derived from other sources, including text entry by users, which may span dietary changes, changes in dietary supplements or medications) to infer potential causal relationships due to voluntary behavior influencing physiology and thereby biological age, health and longevity. A broad range of techniques can be applied, a naive one being to simply consider statistically significant correlations between preceding voluntary behavior changes and measured physiological changes, but formal approaches include Temporal Causal Inference, Granger Causality Analysis, Sequential Pattern Mining and Time Series Analysis for Causal Inference. In addition to performing a penalized multivariate regression for predicting reduction in WDBAA and EBAA using QCB, with penalties such as L1 and L2 on the parameters and hence features used by the model, we also include the vector F as an additional penalty, in order to optimize for the most effective, lowest friction combination of interventions for lowering biological age.

Wearable-ome—A set of behavioral and physiological biometrics that state-of-the-art technology can monitor longitudinally in the general population. The wearable-ome spans both WDBB and WDPB. Behavioral features refer to features which are directly impacted by an individual's actions or habits, such as the frequency and duration of an exercise session. A physiological feature refers to a measure of the individual's physical makeup or function, such as the individual's resting heart rate.

Subendocardial viability ratio (SEVR)—A ratio calculated as the area under the curve of the PPG waveform during diastole, divided by the area under the curve of the PPG waveform during systole. Image included in the appendix.

Normalized Heart Rate Reserve (NHRR)—Normalized heart rate reserve (NHRR) at a specific moment can be calculated using wearable data by subtracting the individual's resting heart rate (RHR) from their maximum heart rate (HRmax) and then determining their current heart rate (HR) at that point in time. NHRR=((HR−RHR)/(HRmax−RHR))*100%. For example, if an individual's HRmax is determined to be 180 beats per minute (bpm) and their RHR is measured as 60 bpm, their NHRR would be calculated as ((HR −60)/(180−60))*100%. Then, if their current heart rate during exercise or any other activity is measured as 150 bpm, their NHRR at that moment would be ((150−60)/(180−60))*100%=75%. Normalized heart rate reserve expressed as a percentage provides a standardized measure of cardiovascular capacity relative to the individual's maximum heart rate and resting heart rate. This metric allows for easier comparison and monitoring of exercise intensity levels across individuals with varying HRmax and RHR values. Wearable devices equipped with heart rate monitoring capabilities provide real-time data on heart rate, enabling users to track their NHRR during various activities and optimize their training regimen accordingly.

Wrist Worn Wearable PSG (WWWPSG)—The term “Wrist Worn Wearable PSG (WWWPSG)” refers to a wearable device designed to monitor physiological signals relevant to sleep staging and quality. This device incorporates both traditional polysomnography (PSG) measurements and wrist-worn wearable technology. Specifically, it collects beat-to-beat interval data from photoplethysmography (PPG), which measures changes in blood volume using optical sensors, and actigraphy data indicating wrist motion. These data streams are fed into a machine learning model, particularly a Long Short-Term Memory (LSTM) deep learning model, which operates on 30-second epochs to predict the user's sleep stage at any given point in time. The sleep stages predicted by the model typically include wakefulness, light sleep, rapid eye movement (REM) sleep, and deep sleep. The LSTM model achieves an average accuracy of approximately 75% per stage, demonstrating its effectiveness in accurately classifying sleep stages based on wrist-worn physiological data. This innovative approach enables non-invasive and convenient sleep monitoring, offering insights into sleep patterns and quality for improved sleep management and overall well-being.

Digital Signal Processing (DSP)—Transformation of low level sensor data from sensors, typically on a wrist worn wearable tracker, consisting of PPG of different wavelengths, into elemental biometric data, such as the start and stop of individual heartbeat intervals, heart rate, steps accrued and cadence of movement, but also oxygen saturation calculated from the amplitude and intensity of light variation caused by mechanical heartbeat waves at the wrist, measured in the red and infrared range at the photodiode sensor on the wearable.

Wearable Biological Age Predicting Models (WBAPM)—WBAPM for predicting biological age and biological age acceleration, where these models use physiological and behavioral biometrics (WDPB and WDBB) as input and have been trained/are iteratively trained on the output models that predict biological age and biological age acceleration from HBD by application of the BAF principal.

Candidate Causal Relationships (CCR)—Candidate causal relationships (CCR) are statistically significant relationships discovered between a voluntary behavior or intervention (such as a lifestyle change, supplement change or medication change) and WDBA or WDBAA. This can be applied either at the population to discover general CCRs as well as at the individual level to discover personalized CCRs. CCRs can be used as communication points with a user to make them aware of potential causal effects of their choices, to help them improve their understanding and thereby management of BAA and improve their biological age.

Optimized Intervention Combination (OIC)—Several changes to lifestyle and interventions are possible. Interactions between interventions with their ability to slow BAA can be complex and with many possible permutations, are largely unknown. Therefore, it is necessary to use data both at an individual level to discover longitudinally as a user adds/drops interventions from their regimen, what combinations work best, as well as at a population level. In addition to using a multivariate approach that considers data on all available behaviors and interventions, the analysis also takes into account the friction associated with a particular change or intervention, in order to obtain the most effectual set of changes for reducing BAA at the lowest level of friction.

### Partial Longitudinal Causality, Composability of Interventions

Some of the challenges discussed above can be overcome by aiming for more detailed and comprehensive outcome measures versus the simpler clinical end-points that are currently in use, like disease status, all-cause mortality or a small collection of health biomarkers like blood pressure or cholesterol. A concrete example of such a comprehensive outcome measure would be to obtain high throughput biology samples, such as a methylome or transcriptome for each participant. This gives an indication of which genes have been activated by a specific lifestyle intervention or drugs. Within these comprehensive outcomes, it is possible to assess the activation of cellular components that are known to causatively and longitudinally extend healthspan where they have been directly bioengineered to have varying activity levels. In this manner, some level of longitudinal causal understanding is achieved, albeit on the partial phenotype and not the complete phenotype of a person undergoing an intervention. This level of causality can be seen as intermediate between a randomized control trial (full causality) and an observational study where only correlative relationships are discovered. By focusing on cellular components that act as master regulator switches that have a large causative downstream domain (i.e. regulate thousands of downstream components), this partial domain of causality can be greatly broadened compared to a random cellular component. Well known examples of such components include AMPK, mTOR and the sirtuins, each of which have been shown to impact longevity directly and universally across a wide range of animal models in this manner. Transcription factors, such as hypoxia inducible factor, which drives increases in aerobic fitness, directly regulate the transcription of hundreds of genes. It would also be possible to assess the activation of cellular components known to have negative effects. By having such a detailed view, it becomes more feasible to investigate which interventions would be complementary by activating non-overlapping beneficial cellular components to achieve a positive outcome more effectively when combined versus the opposite scenario, where two interventions might function via the same cellular pathways and potentially provide a less powerful combination. It might also be possible to attenuate negative side effects of specific interventions, by the addition of interventions that are known to specifically attenuate these at the level of cellular component activity identifiable through high throughput biology samples.

### Cost Reduction Via Remote Administration and Compliance Quantification

Another remaining challenge is that lifestyle programs require costly administrators to assess or enforce adherence, before the impact of the intervention on healthspan can be estimated through mathematical models that map quantified behavioral changes to quantified changes in health. Trials can also not proceed in a fashion where they are ongoing and where participants are monitored remotely and where participants are not necessarily synchronized temporally when participating in an ongoing set of trials that test multiple interventions. This challenge can be overcome by providing IOT technology, such as wrist worn wearables and mobile phones, to track adherence to lifestyle choices. Wrist worn wearables can detect actualized behavioral changes in response to an intervention program provided (possibly via mobile phone)—simple examples include exercise duration and intensity as well as sleep amount and timing of going to bed each night. This technology also allows lifestyle intervention studies to be taken from the clinical trial environment to everyday living, as it becomes possible to provide intervention programs in an ongoing manner, while quantifying changes in behavior in line with these recommendations. Since partial compliance to a program can then be quantified, full adherence and enforcement through trial administrators, is no longer required. Sham lifestyle interventions can also be provided to randomized control groups to convert this into the equivalent of a randomized controlled trial.

### Personalization Through Epigenome Samples and Wearables

Another remaining challenge is that insights based on the study designs described earlier, are generated at a population level, rather than at an individual level. With the description provided earlier, IOT based devices that monitor compliance in combination with high throughput biology samples that are taken periodically to assess the functional state of cellular components, provide a mechanism for discovering causal mechanisms and to quantify the strength of different known causative mechanisms at the level of individual participants. For example, fasting results in body wide changes in gene regulation. The upregulation of AMPK transcription or the epigenetic modification via methylation that controls its transcription, can be measured periodically through white blood cells using blood samples and compared to interventions in diet, sleep and fasting using an IOT system that tracks compliance via wearable devices as well as smartphones and nearable devices for keeping track of diet, medication or supplement intervention. Individual variability in how strongly AMPK becomes activated by a fixed intervention, e.g. 16 hour fasting, will vary across individuals and this information can be aggregated through transcriptomic and methylomic data gathered over successive interventions measured by IOT and taking periodic measurements of cellular components, in order to provide a personalized fasting window that achieves an intended level of AMPK activation and healthspan extension.

Beyond the use of IOT, such as wearable devices, to track compliance to lifestyle intervention programs in the proposed invention, these devices can also measure continuously several physiological processes that are indicative of increased or decreased healthspan. For example, heart rate variability (HRV), Pulse waveform (PWF) and resting heart rate (RHR) each contain information on participant health. These health measures, alongside several others, such as wearable derived hypnograms, can be used to get an integrated view of the higher level functional state of an individual. Functional measures of health that can be estimated from wearables, with VO2max also being a good example, provide a second layer, beyond molecular components, for assessing the integrated outcome of an intervention in an IOT environment. Such integrated assessments can be used to confirm whether the partial causal information derived from molecular changes are producing higher level functional changes, such as increased VO2max or increased cardiovascular health.

Finally, changes in both molecular components measured through high throughput biology samples, as well as physiology, as measured through IOT technology, can be combined into single indices, called biological ages. Molecular clocks for measuring not chronological, but instead biological age, such as the Horvath clock are known in the scientific literature and can be used to determine whether, from an epigenetic point of view, a participant is accelerating or decelerating their overall biological age. The same concept can be extended to wearable biomarkers that measure human physiology. By considering the impact of combinations of interventions on an individual, it becomes possible to estimate and optimize their joint long term effectiveness for a particular individual.

In summary, the disclosure is directed at systems and methods that includes wearable and other IOT technologies for monitoring human behavior and physiology, alongside periodic measurements via high throughput biology of changes in cellular component activity in response to interventions that are trialed under free-living conditions to extend human health span. Knowledge about the long term causative impact of master regulator cellular components, such as AMPK, mTOR and the sirtuins, is used to estimate partial long term causal impact on healthspan of interventions, which are trialed dynamically through the ability of IOT and wearable technology to measure trial compliance in the field. The system takes the process of discovering causal interventions from the clinical trial environment, to the consumer environment, to dramatically scale up the rate at which effective interventions are learned, where interventions include, but are not limited to dietary, supplementation, pharmacological, sleep and exercise.

Functional biomarkers of aging are biomarkers which provide insight to organ or system function with regards to aging. Grip strength, a proxy for muscular function, and VO2 max, the maximum capacity of the muscle to utilize oxygen during exercise, have been shown to decrease with age. These biomarkers, along with lung function, have been included in epigenetic clock construction. Structural changes to blood vessels, measured by changes in pulse waveform (PWF) features, have been shown to change with age and overall vascular health status.

Wearable devices are strong candidates to measure the functional biomarkers of aging in a frictionless reliable way. Photoplethysmography (PPG) is a non-invasive method to measure changes of blood volume in tissue. Typically, a light emitting diode (LED) is shone onto the skin and is absorbed by the blood and tissue, and the remaining light is reflected and measured by a photodiode. As the heart beats, blood volume in the tissue changes, causing the corresponding light which is reflected to change. Once this signal is processed, biological data, such as the PWF, heart rate, heart rate variability (HRV) and blood oxygen levels can be extrapolated. In addition to PPG, an accelerometer captures sedentary and activity data and has been used to capture such data from large population cohorts. Accelerometer based devices have been used to measure biological aging, showing an association with activity levels and age acceleration, but currently PPG based biometrics have not been incorporated in biological age measurement. LifeQ Inc., a wearable device biometric company, has the capability to produce a wide range of wearable-based biometric features. We define the wearable-ome as the set of behavioural, an individual's actions or habits, circadian, a characterization of sleep habits, and physiological, a characterization of cardio and peripheral vasculature, biometrics. Mapping the wearable features to a molecular measure of biological age provides non-invasive, easily accessible age monitoring.

The various inputs that wearables can capture are provided below.

### Wearable Derived Physiological Biometrics (WDPB)

Pulse waveform derived features (PWFF) including:

PPG Crest time—The absolute time from the starting point of PPG (O) to systolic peak (S).

Augmentation index—The height of the systolic peak (S) over the diastolic peak (D).

Pulse wave velocity—Pulse wave velocity can be measured with devices such as the Sphygmocor by recording the time that it takes for the mechanical pulse wave to traverse a section of the large arteries between the carotid and femoral or brachial and femoral arteries. It is correlated to elementary pulse waveform features such as the PPG rise time and the timing and relative amplitude of the diastolic and systolic peaks and could also be fitted using machine learning approaches that automatically produce features such as convolutional neural networks.

Large artery stiffness index (LASI)—The stiffness index is the subject height divided by time difference between systolic (S) and diastolic peaks (D):

$$S I = \frac{height}{Δ T}$$

Systolic duration—The time difference between the starting point of PPG (O) and the dicrotic notch (N).

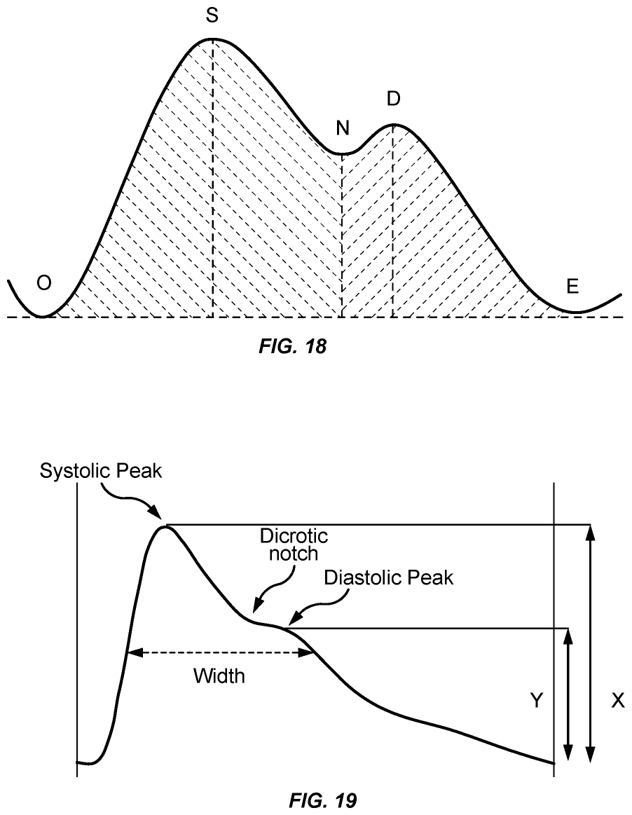

Coronary circulation estimate: Subendocardial Viability Ratio (SEVR)—SEVR is defined as the ratio between area_NE and area_ON, as shown in FIG. 18. Subendocardial viability ratio (SEVR) is ratio between the area under the curve between N to E point(green dash region) and the area under the curve between O to N point (red dash region).

Systemic Vascular Resistance (SVR)—the estimate through pulse height, as shown in FIG. 19. A typical waveform of the PPG and its characteristic parameters, whereas the amplitude of the systolic peaks is x while y is the amplitude of the diastolic peak. According to Awad et al. (2007), the relationship between the PPG waveform and systemic vascular resistance has been explored extensively. Specifically, the study discusses the correlation between the two variables in detail (Awad et al., 2007). The author defines the pulse width as the pulse width at the half height of the systolic peak. They have suggested that the pulse width correlates with the systemic vascular resistance (SVR, Peripheral vascular resistance) better than the Systolic amplitude, as shown in FIG. 19. As mentioned in the last section, the precapillary arterioles are also one of the reflecting sites generating the reflecting wave. The arteriolar system represents a sort of narrowing of the arterial system. The forward wave breaks against the “wall” made up by peripheral vascular resistance, then the backward wave is going to superimpose on the forward wave, which is similar to the backward wave caused by the apparent site of reflection. So the complexity of pulse waveform increases again. The peripheral arteries, such as femoral, brachial and radial arteries, are closed to the main site of wave reflection, so that the superimposition of the forward and backward waves occurs during the systolic phase,

Blood pressure index (BPI)—This can be derived from features of the critical points of the pulse waveform. The specific ratio is the b/a ratio and is outlined in several studies (Zhang et al., 2018; Takazawa et al., 1998; Yao & Liu, 2020).

Drops in oxygen saturation during sleep, wearable oxygen desaturation index (WODI)—The Oxygen Desaturation Index (ODI) is calculated of the total number of oxygen desaturation events during core sleep per hour. Plot shows daily ODI value. Oxygen saturation is measured using the AC/DC/AC/DC ratio of ratios of infrared PPG and red PPG channels. For wearables, drops of 7% empirically were the best capable of separating those with moderate-to-severe sleep apnea from those that are normal or have light sleep apnea.

### Heart Rate Features:

Resting heart rate (Gil et al. 2010)

Maximum heart rate (Huang and Selvaraj, 2020)

Beat to beat intervals

Heart rate variability (Gil et al. 2010

Arrhythmia and ectopic beat propensity (AEP) (WO 2022147271 A1)

Multiwavelength features

SpO2 (Yang et al., 2015)

VO2max estimates—Estimating VO2max from wearable data involves a combination of behavioral, physiological, and demographic factors. Here's a high-level description of how this can be achieved:

Behavioral Aspects: Frequency, Intensity, and Duration of Exercise: Wearable devices can track exercise sessions, including the frequency, intensity (heart rate zones or perceived exertion), and duration of each session. These data help establish the individual's exercise habits and training volume.

Physiological Aspects: Resting Heart Rate (RHR): Resting heart rate, measured during periods of inactivity, provides insight into cardiovascular fitness. A lower RHR often correlates with higher aerobic fitness levels.

Pulse Waveform: Analysis of pulse waveform data, captured using photoplethysmography (PPG) sensors in wearables, can provide additional information about cardiovascular health and function.

Demographic Information: Age, Height, Weight, and Gender: These demographic factors are essential for estimating VO2max using predictive equations, as they influence aerobic capacity. Maximum Heart Rate (HRmax): Measured or estimated HRmax is another crucial parameter used in VO2max estimation algorithms.

Laboratory Test Results Sharing: Users have the option to input or share their laboratory-tested VO2max results if available. This information serves as a reference point for the wearable device to calibrate its estimations based on the user's physiological data.

Running Speed via Phone GPS: Some wearable devices can utilize built-in GPS or connect to a smartphone's GPS to track outdoor running speed. Running speed, along with heart rate data and other factors, can be used to estimate VO2max.

Combining these data points, wearable devices employ algorithms and predictive models to estimate VO2max. These models often consider factors such as exercise intensity, heart rate response, demographic information, and sometimes additional parameters like stride length and running economy. Users can access their estimated VO2max through accompanying smartphone apps or web interfaces, providing them with insights into their aerobic fitness levels and tracking their progress over time. It's important to note that while wearable-based estimations can provide valuable insights, laboratory-based testing remains the gold standard for accurately measuring VO2max.

### Sleep Features (Based on WWWPSG)

Deep sleep drive (deep sleep minutes relative to total sleep time) (Birrer et al., 2024)

REM sleep drive (REM sleep minutes relative to total sleep time) (Birrer et al., 2024)

Total sleep drive (total sleep time within the context of scheduled wake time)

Circadian drive (propensity toward advanced or delayed circadian rhythm within the context of scheduled wake time) (Birrer et al., 2024)

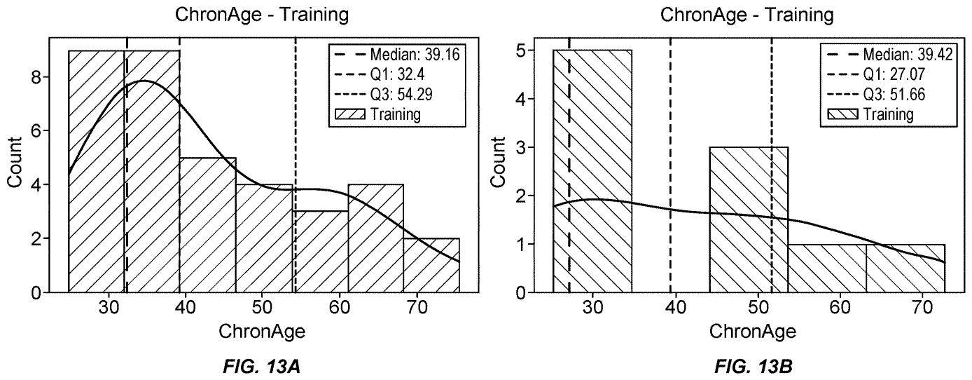

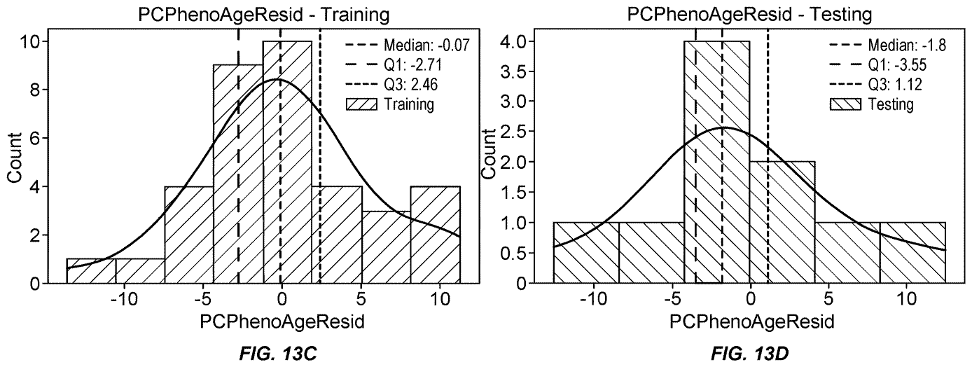

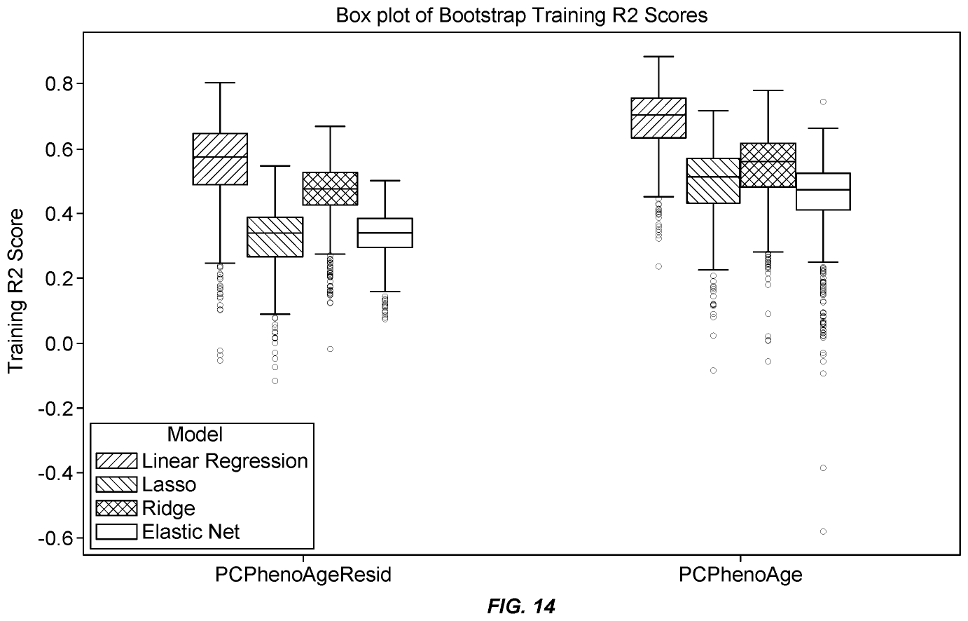

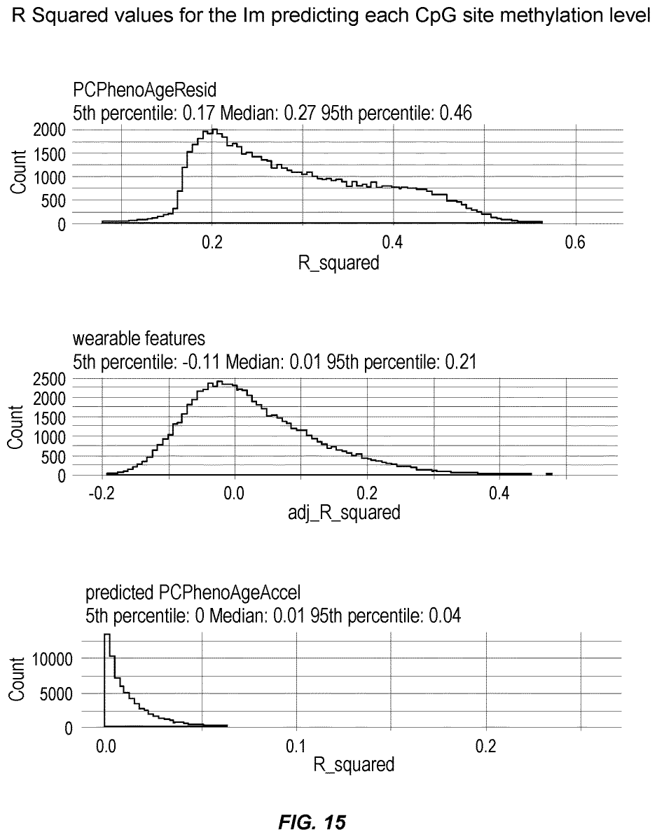

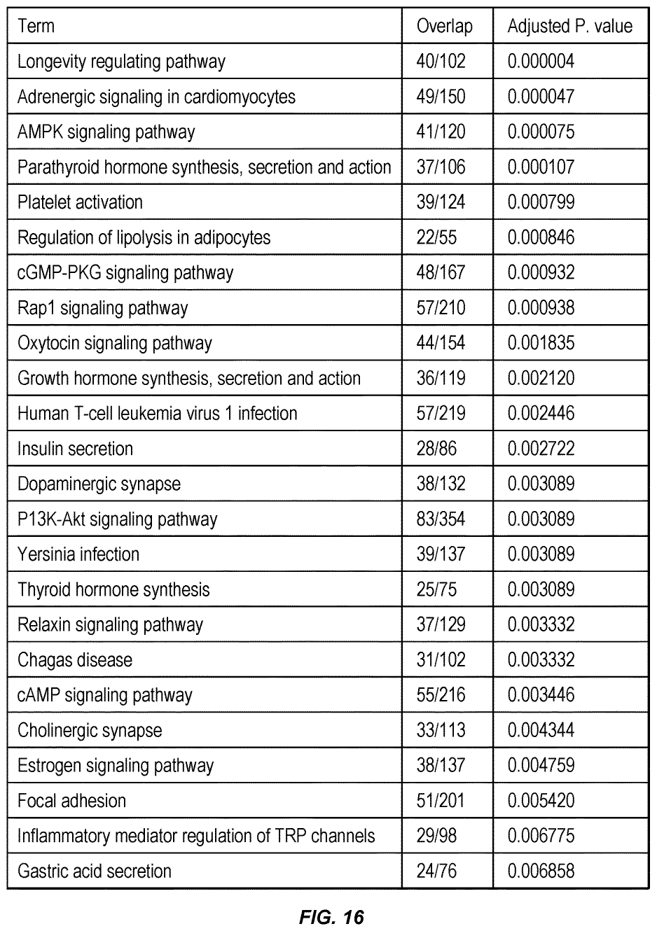

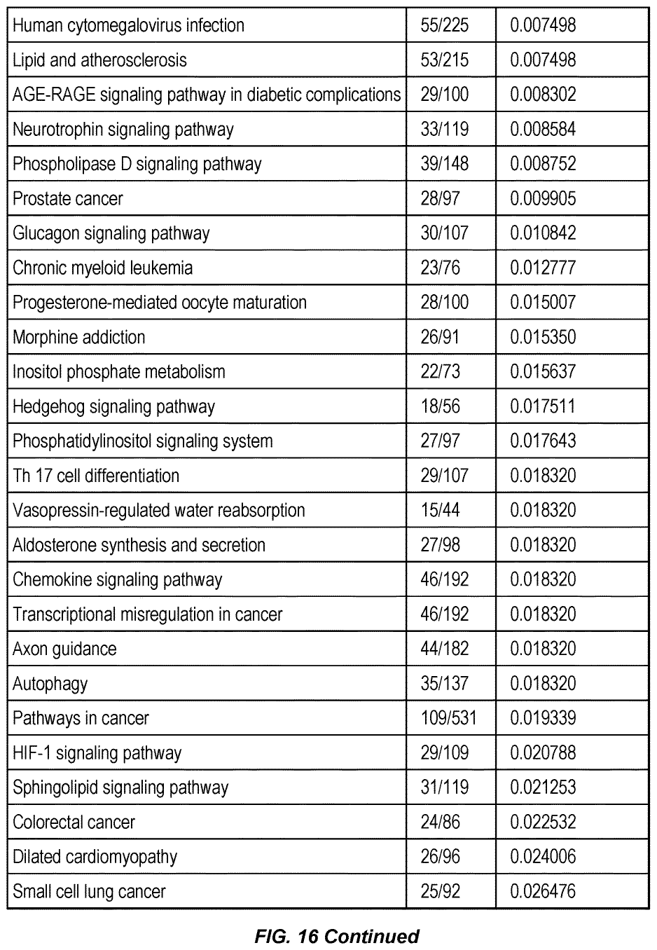

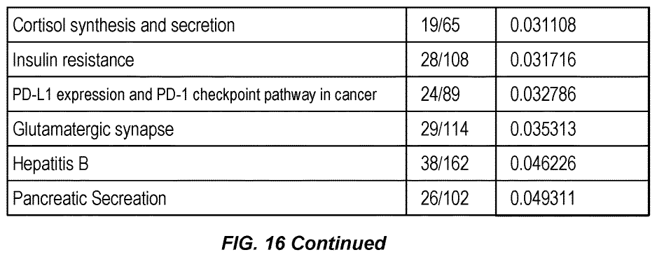

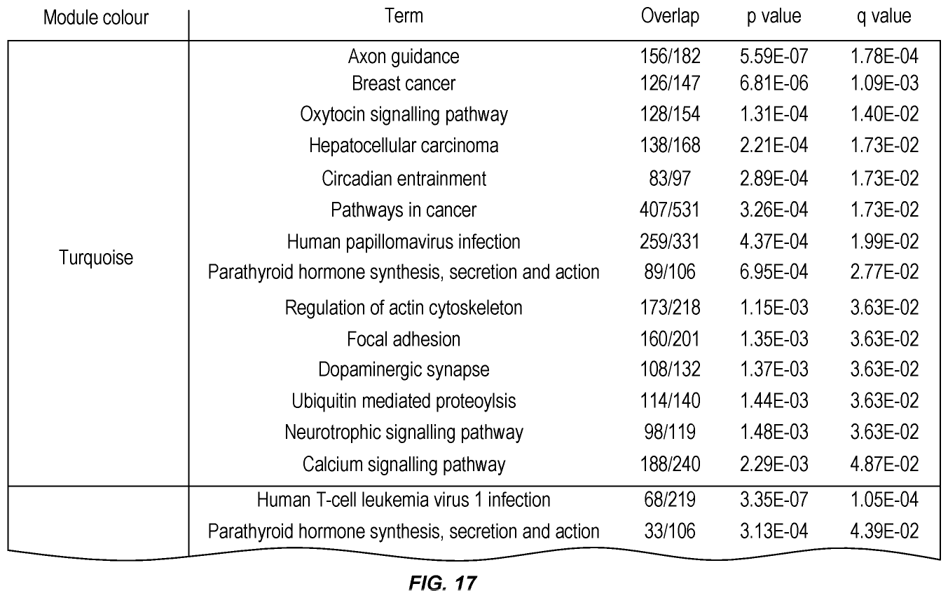

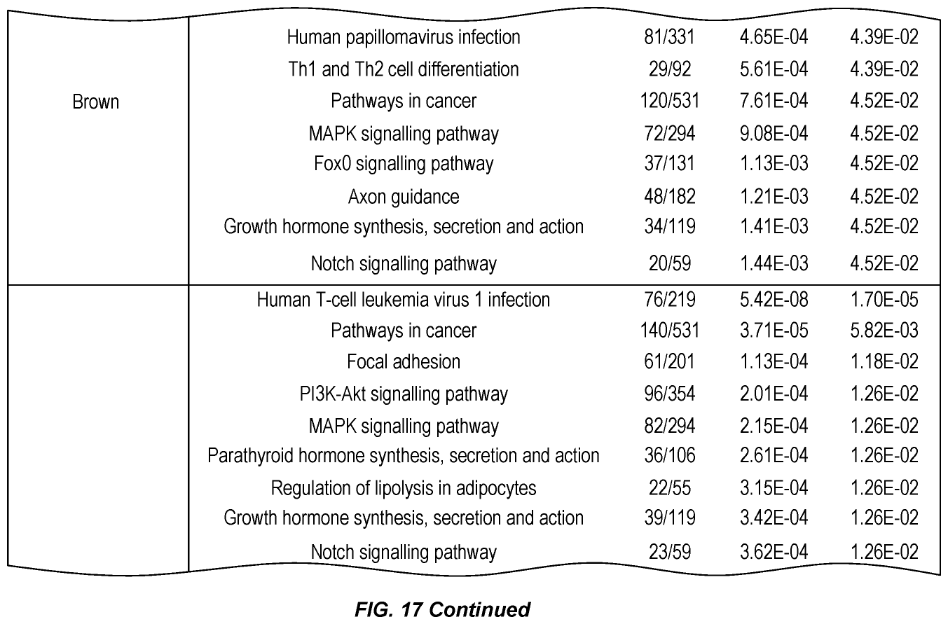

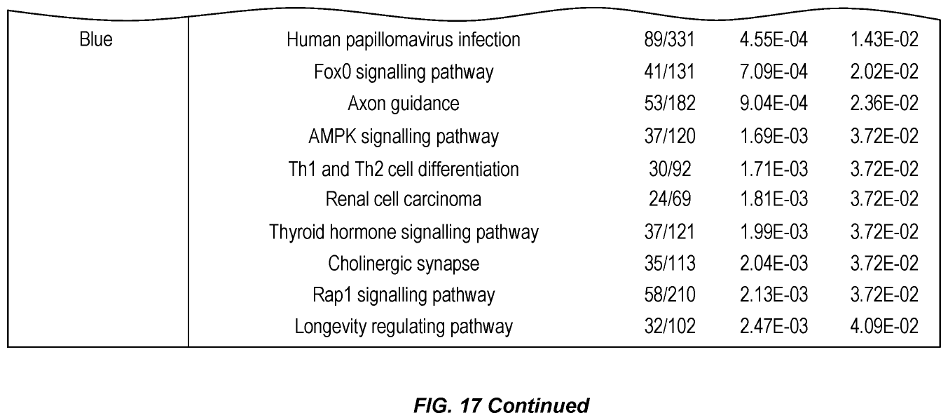

Latent dimensions that combine several sleep features, such as principal component analysis (PCA) (See FIGS. 13A-13D)

Sleep onset latency (Birrer et al., 2024)

Amount of Wake After Sleep Onset (WASO) (Birrer et al., 2024)

### Wearable Derived Behavioral Biometrics (WDBB)

Exemplified by, but not limited to the following:

### Sleep Features

Variability in when a user goes to bed (Birrer et al., 2024)

Timing of when a user goes to bed (Birrer et al., 2024)

Reduction in total sleep time within the context of scheduled wake times (Birrer et al., 2024)

### Activity and Exercise

Vigorous exercise minutes as defined by NHRR (Birrer et al., 2024)

Moderate exercise minutes as defined by NHRR (Birrer et al., 2024)

Light exercise minutes as defined by NHRR (Birrer et al., 2024)

Sedentary time (Birrer et al., 2024)

Steps per day (Birrer et al., 2024)

Timing of exercise with respect to the circadian rhythm (Birrer et al., 2024)

Sleep timing and duration (Birrer et al., 2024)

### Additional Behavioral Biometrics (ABB)

Additional behavioral biometrics recorded from any other applicable IOT device capable of transmitting lifestyle behavioral information

Daily nutrition, exemplified by mobile apps keeping tracking meal data

Water intake

Screen time

Light exposure patterns throughout the day

Stress levels

Social connectivity

Mental health

Lifestyle related annotations provided by users through phone apps to indicate interventions like fasting

### Additional Physiological Biometrics (APB)

Additional physiological biometrics recorded from any other applicable IOT device capable of transmitting lifestyle behavioral information

IOT weight scale for recording weight

Mobile phone, through integration with health data aggregated in compliance with HIPAA from other health platforms like Apple Healthkit

Motion and orientation from motion capture of users' head and body position in VR (virtual reality) and AR (augmented reality)

Rankings, reaction time or other neurocognitive performance metrics from computer gaming platforms

Nearable devices

### Wearable Predictions of Epigenetic BAA and BA (WDBA and WDBAA)

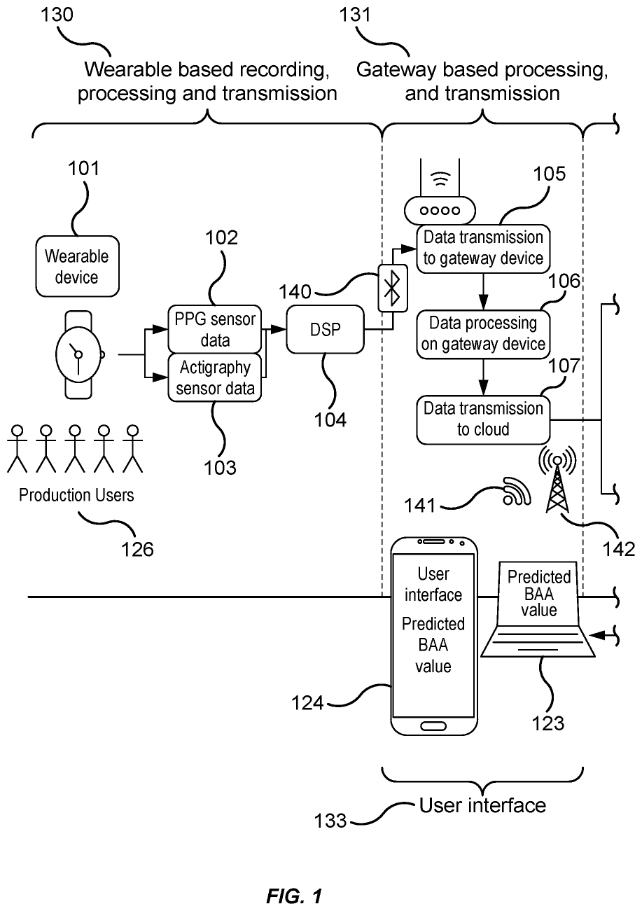

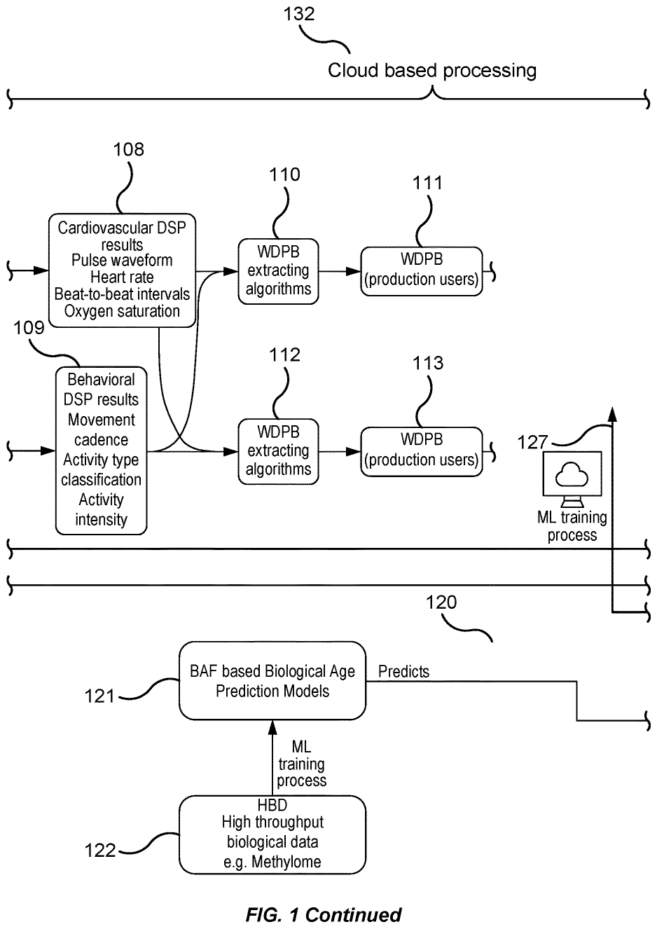

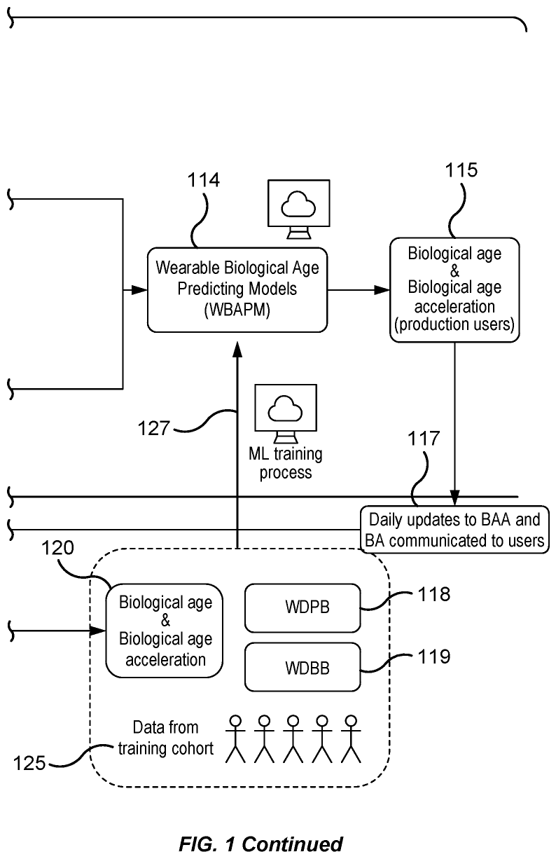

In an aspect, the invention consists of a network of physical components that runs different software algorithms. As shown in FIG. 1, these may include a wearable device (130), typically, wrist worn, similar to a smartwatch or body monitor (101), capable of obtaining raw sensor readings for PPG(Photoplethysmography) (102) and actigraphy (103) and running elementary DSP to extract low level biometrics. The system can also include a gateway device (131) that can communicate with the wearable via one channel such as Bluetooth and other communication means (140) and to the cloud through an appropriate channel (107) such as a home wifi network (141) or 5G data transmission (142). The system can also include a means for extracting the biometrics (110, 112) and running the models predicting BA and BAA from the wearable-ome (114). This can be done on a cloud service like AWS (132). There can also be an interface for communicating the results to an end user (133), such as a cell phone display or audio (124). The system may also include a means for training (127). The means can utilize a BAF based model outputs (121) and matched WDPB (118) and WDBB (119) data, a wearable-ome biological age predicting model in the cloud (114), that can predict BA and BAA (115) from the wearable-ome (111, 113), such as a physical computer or cloud service (e.g., AWS sagemaker). The system can also include a means for using the model above to make BAA and BA predictions (115) using wearable-ome data from production data from users in the production environment wearing wearable devices generating data from which WDPB (111) and WDBB (113) are produced as inputs to 114.

The invention comprises several data processing and transmission steps. Analog sensor data is digitized (via an ADC) from the analog sensor readings of PPG (102) and Actigraphy (103) sensors on a wearable device (101) with a microprocessor and data transmission capabilities, such that the PPG is in close contact with the skin of a user of the system. This creates a stream of PPG data, sampled at a frequency of 25-50 Hz, typically with a wavelength of 525 nm. Actigraphy is sampled typically in all three axes, x,y an z and at the same frequency as the PPG signals, such that adaptive filtering can be applied to remove motion artifacts from the PPG signal as part of it's DSP chain (104, 106) prior to producing cardiovascular DSP results (108) and behavioral DSP results (109). These intermediate results for cardiovascular DSP results (108) consist of

Pulse waveform segments gathered under low motion and high signal quality conditions, as well as representative pulse waveform signals, which are averages of normalized (unit duration and unit amplitude) individual pulse waveforms, typically collected during 30 second recording. Signal quality is determined through consistency of waveform signals, SOTA approaches like comparing the consistency of basic waveform features such as amplitudes and durations.

Heart rate predictions, 1 Hz, which are obtained through the use of frequency domain based algorithms and adaptive filters that make use of the accelerometer data in conjunction with the PPG to predict heart rate, a technique covered in SOTA (Huang and Selvaraj, 2020)

Beat-to-beat timing, where we have a timestamp for when a beat started as well as a duration value, indicating the time between two successive heartbeats.

Optionally oxygen saturation, 1 Hz. This is computed as found in SOTA, where the ratio of ratios is used for infrared and red PPG channels

The intermediate features for behavioral DSP results (109) consist of

Aggregated accelerometer activity (e.g. magnitude of the 3D triaxial jerk vector on the accelerometer averaged over intervals at various scales, e.g. 1 Hz)

Cadence, through Fourier analysis of the raw accelerometer signals and in the presence of an activity type (3 below) with cadence
1.) Activity type classification (based on supervised machine learning algorithms of the accelerometer signals, classifying walking, running, sedentary, rowing, cycling states)
2.) Activity intensity and activity minutes—cardiovascular intensity via Heart Rate Reserve (HRR) producing light, moderate or vigorous intensity bands with their duration measured as activity minutes (Muggeridge et al., 2021)
3.) Fall triggers—brief, anomalous, high acceleration events
4.) Step counts—steps per day, steps per activity
5.) Activity events—aggregation of periods that are mainly of light, moderate or vigorous intensity (Muggeridge et al., 2021)
6.) Sedentary events—non-active periods
7.) Fitness events—events of aggregated intensity that exceeds moderate or vigorous intensity based on HRR

The DSP results (108 and 109) are used for extracting higher level biometrics, which are more closely tied to human behavior and physiology than the more raw DSP results. The individual elements of the WDPB and WDBB are listed within their definitions. Calculating each of these features is beyond the scope of this document, literature references are provided to add the necessary specificity and enablement. Select features are also described in the appendix.

The wearable-ome (111 and 113) biometric feature set is used in the next step of the process as inputs to the WBAPM (114). The wearable-ome features are aggregated in time, such that each is a representation of the median value of the feature over the course of the past 40 days to produce a stable noise free value, conditional to the necessary volume of data (>60% of the 40 days has >70% coverage per day) being available.

The WBAPM model (114) produces estimates of BA and BAA (115) for production users (WDBA and WDBAA) from said aggregated features, which are stable and which can be updated on each calendar day (117) with the addition of the newest day's wearable-ome biometric data considered as part of the 40 day buffer for recalculating the median value for each feature. The daily BAA and BA predictions (117) are subsequently relayed to the same users on whom data was collected (126) via a user interface (123 or 124) to provide feedback on their estimated biological age based on the BAF principle, to help them discover whether their current interventions are helping or hurting their efforts to reduce age acceleration. At this point, users are annotating interventions themselves and are comparing changes in their age acceleration or deceleration with these interventions as well as their behavior as quantified and also communicated to them via a user interface (123, 124), not indicated in FIG. 1 to limit visualization to the core aspects of the invention.

Training of the WBAPM model (114) involves the creation of a machine learning model. Simple linear regression approaches with regularization like ridge regression provided an ability to predict 30-50% of variation (Pearson R2) in epigenetic age acceleration in a training cohort of 48 individuals tracked over 40 days with wrist worn wearables capable of generating wearable-ome biometrics (111, 113). The input features to the training process was again the 40 day median values for wearable-ome biometrics and the paper describes feature selection in more detail. Physiological biometrics (WDPB) were much more strongly correlated to BAA and BA than behavioral metrics over this timescale (WDBB).

Association of Behavior/Interventions with WDBAA

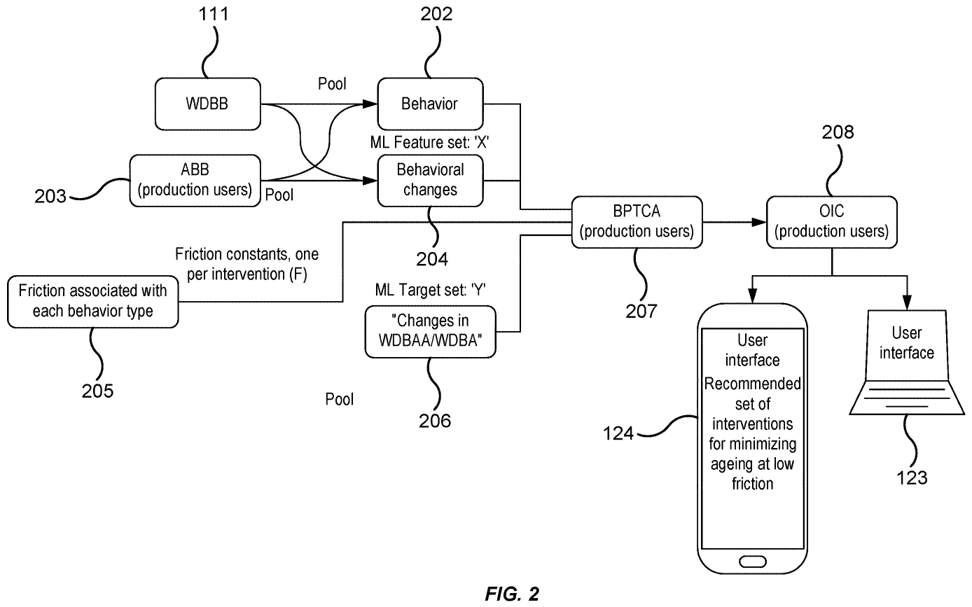

As included in the definition of BPTCA (207), several quantitative frameworks exist for detecting causal relationships between random variables. In a simple example, training a supervised machine learning model to predict changes in WDBA, using WDBB and ABB from matching individuals over the course of data collected for them, allows one to evaluate through performance metrics and feature importance of such a model, which set of features (behaviors) are required at a minimum to achieve most of the WDBAA lowering effect. Introducing a lag period for the WDBA, ranging on the same timescale as physiological changes (1-30 days for exercise), can also help to look for results that follow changes to WDBAA. Performing such a fit on population data, has clear potential to highlight causal relationships and help to generate evidence automatically with regards to which diet, exercise, nutritional and pharmacological interventions, most successfully combat BAA. As per the example of using a supervised learning model to predict changes to WDBAA (206) from WDBB (111) and ABB (ABB), we have labeled the features as X and the target as Y in FIG. 2. An additional aspect to the invention is the addition of a weight that resembles the weight of a particular intervention.

Let's assume we have
X as the matrix of intervention/behavior features (columns) with samples from various individuals collected at various time points in the rows.
y as the vector of WDBAA values, but lagged by a prespecified period compatible with physiological changes in response to healthy behavior (e.g ranging between 1-30 days)
B as the vector of coefficients to be estimated for the model
λ₁ and λ₂ as the regularization parameters for L1 and L2 penalties, respectively
F as the vector representing the additional friction penalty for each feature

The optimization equation can be written as:

$${min}_{β} ( {‖ y - X β ‖}^{2} + {λ}_{1} {‖ β ‖}_{1} + {λ}_{2} {‖ β ‖}^{2} + {\sum }_{i = 1}^{p} {F}_{i} {β}_{i} )$$

Where:
∥y−Xβ∥² represent the squared loss function (least squares)
λ₁∥β∥₁ represents the L1 regularization penalty (Lasso)
λ₂∥β∥² represents the L2 regularization penalty (Ridge)

$${\sum }_{i = 1}^{p} {F}_{i} {β}_{i}$$

represents the additional penalty term, where Fi is the friction constant associated with the i-th feature and Bi is the coefficient corresponding to that feature.

By working with normalized feature values in X and selecting the features that retain large enough Bi values to still contribute to the prediction, it becomes possible to select a subset of interventions that have the most meaningful impact on WDBAA at the lowest friction, thereby eliminating interventions that are redundant and those that are high friction without adding benefit above low friction features.

This process produces what is defined as OIC (208) in this document, which provides the users with an optimized intervention plan (123, 124) that can be further tailored based on the relative friction that the user perceives as being associated with each intervention, hence the user can make adjustments to F to obtain personalized results. The user can also add criteria that limit the search for an OIC to other users of similar demographics such as age, BMI and sex, to further narrow down and personalized a set of OIC's and finally the user can also weight their own dataset, which could span multiple interventions as part of the OIC generation, to weight the OIC more towards personalization with the necessary caveats on low data volumes for one user.

### The Wearable-Ome as an Interpretable Bridge to HBD BAA and EBAA

HBD provides a way of calculating BAA that is most capable of measuring chronological aging at a biological level Horvath (2013) and is also the forensic standard in estimating chronological age for human samples. As mentioned in the introduction, it is also significantly related to human health, disease and risk of mortality. At the same time, corruption of epigenetic information is arguably the strongest hypothesis currently for explaining the underlying causes of aging. BAA derived from HBD such as EBAA however, is not very interpretable, aside from the compound, one dimensional index value of EBAA in years (difference between biological and chronological age).

Due to the shared variance demonstrated between WDBAA and EBAA, in the range of 30-50%, based on simple linear predictors on a small pilot dataset of 48 individuals tracked for 40 days, the wearable-ome provides a much more interpretable bridge for understanding BAA as measured through HBD compared to EBAA. This statement is based on the assumption that the FCC of the ~23,000 genes in the human genome is much less interpretable or accessible to the general population than the features of the wearable-ome which include common lifestyle related behaviors such as sleep and exercise levels as well as physiological measures (pulse waveform, heart rate, heart rate variability, arrhythmia, oxygen saturation), which although more in the clinical domain and less interpretable than behavior, are still much more relatable than FCC for genes in the human genome.

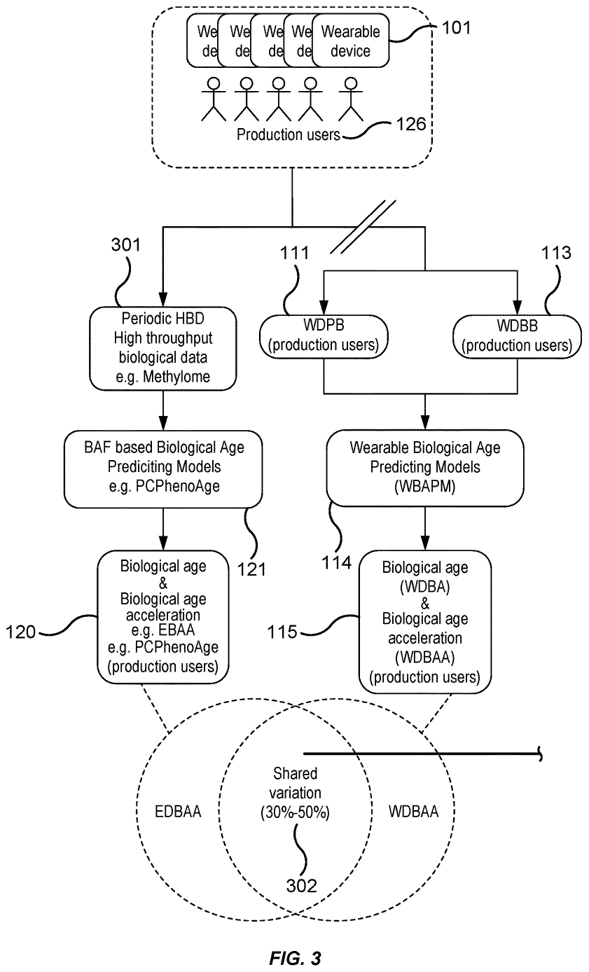

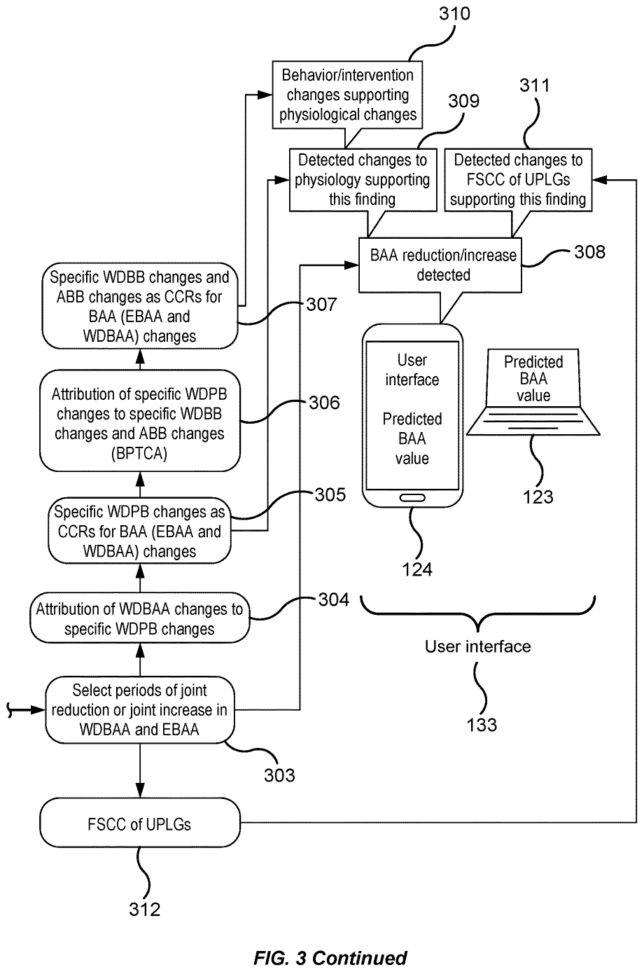

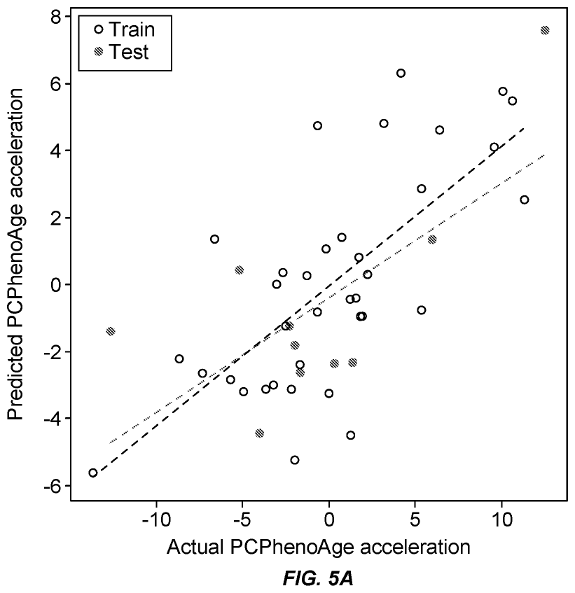

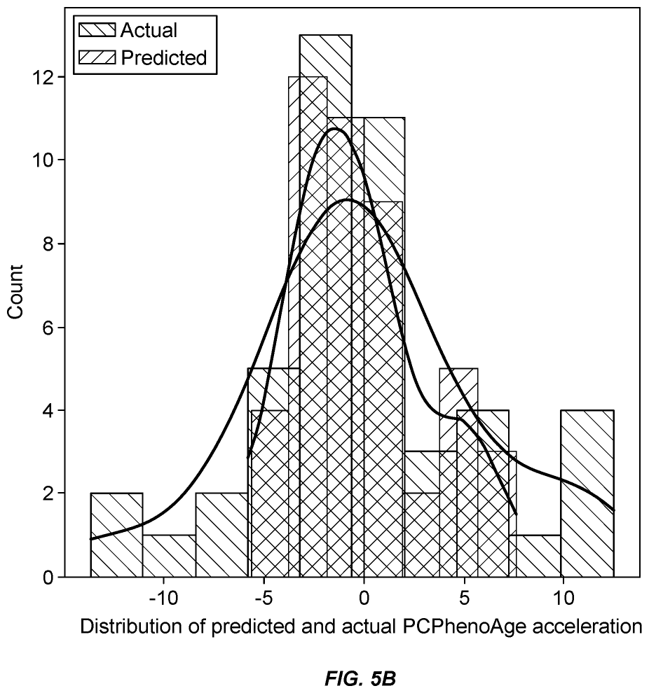

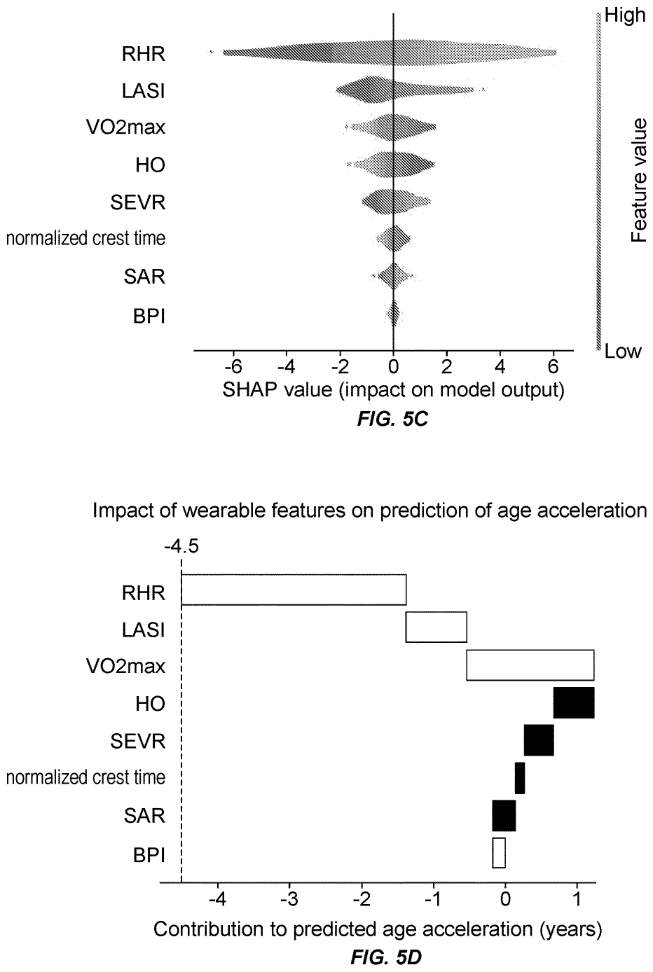

The proposed invention also includes in FIG. 3 the ability to relate changes in HBD BAA, such as EBAA, to changes in WDBAA (115) in order to make it more interpretable to the user. To unpack this further, in FIG. 3, the shared variation (302) is represented in a simple Venn diagram. Periods where both WDBAA and EBAA indicate an increased BAA, or both indicate a decrease in BAA are selected (303). For these periods, the goal is to communicate to the user potential causes for the reductions or increases in aging observed. For the WDBAA (115), as described in the UM, the age acceleration or deceleration can be interpreted in terms of the WDPB changes through Shapley values (304), see FIG. 5C. These values are additive ways of expressing feature importance for a model based on how different features of the training data influence the overall prediction, but the Shapley analysis can also be used to determine the relative contribution of individual features toward the WBAA for a single user (305), thereby highlighting the most significant physiological changes that resulted in the increased or decreased WDBAA (303). In such an analysis, features that are at the training population mean level, would for example carry zero weight toward the overall prediction, while those that deviate most from the mean and carries the highest Shapley predictive power, are the ones that would have the highest predictive power when we consider a single WDBAA datapoint on one user.

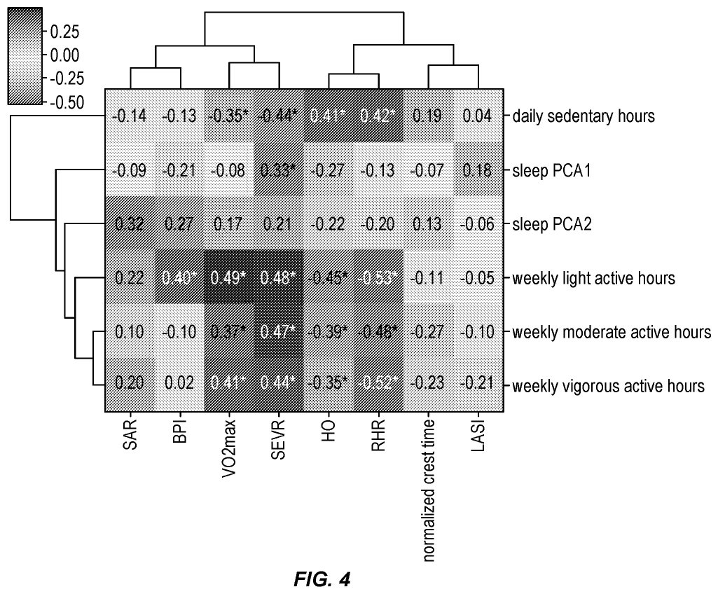

FIG. 4 illustrates statistically significant relationships between WDPB (columns) and WDBB (rows) are visualized. This makes it possible to also relate the specific WDPB changes (305) that underlie the WBAA changes (302) to specific WDBB changes (307) (shown in FIG. 4) and the correlations in FIG. 1 support the viability of using BPTCA.(306, FIG. 2 207), to extract such relationships. The outcome, of this chain of processes, is the set of potential WDBB changes (307) that underlie the detected EDBAA and WDBAA, as well as the set of specific WDPB changes (304 that potentially underlie the EBAA and WDBAA). With these two sets, we can communicate to the user (308, 309, 310) the behaviors that are potentially increasing or decreasing the detected changes to their BAA by means of WBAA and EBAA, as shown in FIG. 3.

Finally, more can be done leveraging periodically collected EBAA (120) and the FCC of various genes in the HBD for the epigenome. It is possible to disclose changes to the FCC of a small set of UPLGs. This makes it possible to communicate (311) to the end user, changes to the predicted activity of key longevity regulating genes (e.g. AMPK, mTOR and the sirtuins), to help users understand how their behavior is impacting the activity of these pathways, either to accelerate or decelerate aging.

### EXAMPLES

This study aims to utilize wearable data to predict biological age and age acceleration, and extrapolate information derived from CpG methylation. This was achieved by recruiting individuals of various ages and sampling wearable and blood data. The hypothesis is that wearable features can predict biological age, age acceleration and underlying biology.

### Methods

### Participants, Recruitment, and Study Design

Following ethics approval (Pharma-Ethics 211224438), participants were recruited to participate in the study, recruitment methods are available in appendix. Each participant was supplied with a LifeQ-enabled wearable, utilizing 25 Hz PGG and tri-axial accelerometer sensor signals. Participants completed 40 days of free-living, to which at the end blood was drawn and wearable data collection terminated. Thereafter signal quality filtering and feature generation occurred via LifeQ's device-enablement software libraries and then uploaded into LifeQ's cloud environment. Study researchers were given randomized research identification codes which associated with the study ID number of the participant to map the wearable data to the blood-derived and clinical data. All data were anonymized and stored on Amazon Web Services simple storage service (AWS S3) in compliance with local data privacy legislation.

### Wearable Features

Wearable features were then separated into behavioural, circadian, and physiological features. A “behavioural feature” implies that an individual can alter the feature value based on their conscious decisions. Physiological features are features which give information about the physiological state of the participant and cannot be directly regulated by conscious decision making. Sleep metrics were classified as circadian features. Detailed description of wearable features is illustrated in table 1.

### Blood Collection and Methylation Analysis

Participants were instructed to be in a fasted state and blood was drawn by a trained phlebotomist, at a local pathology laboratory. EDTA tubes were used in the sample collection, which was immediately put on ice before being transported to the contracted molecular laboratory to perform methylation analysis. DNA from whole blood preserved in EDTA tubes was extracted using the MagMax DNA Multi-Sample Ultra 2.0 along with the KingFisher Flex Purification system. DNA quality was assessed using a Nanodrop for DNA concentrations, as well as the A 260/280 ratios.

The Qubit BR dsDNA system was used for measuring dsDNA concentration. The Infinium HD workflow was used for processing the samples (https://www.illumina.com/Documents/products/workflows/), which included multiple steps to prepare the DNA through amplification, fragmentation, precipitation, resuspension, hybridisation, washing, and finally staining of the bead chip for imaging on the iScan instrument. The EPIC chip (Illumina, San Diego, CA, USA) requires an additional bisulphite conversion step prior to the Infinium workflow, which was performed using the EZ DNA Methylation Kit (Zymo Research). Detailed description of the protocol can be found in the appendix. Analysis of the raw methylation data returned from the contracted laboratory was done using the R software v4.2.2. The Chip Analysis Methylation Pipeline (ChAMP) package was used to process the raw red and green .idat files, extracting and normalizing beta values using the BMIQ method.

### Biological Age Calculation

The principal component (PC) versions of multiple DNAm ages were calculated using open-source libraries provided by Higgins-Chen and Levine et, al. Calculated DNAm ages were refined versions of Horvath, Hannum, GrimAge and PhenoAge. Age acceleration was defined as the residual between the linear model of biological age regressed onto chronological age.

### Sleep PCA

Principal component analysis was performed on the sleep features to reduce dimensionality of the feature set. Sleep features were assessed for a high degree of collinearity among features, with highly correlated features being removed. A threshold of 0.15 was implemented on the explained variance ratio to select the number of principal components. Feature loadings were calculated as the square root of the explained variance. A threshold of 0.75 was used to select features of importance to the principal component.

### Overview of Model Development

The development of linear models involved several steps. The data was split into physiological, behavioural, and circadian features. The cohort was split into training and test datasets by an 80:20 (36:10 samples) ratio and stratified according to PCPhenoAge acceleration to maintain a similar distribution in both training and testing datasets (appendix S2). Four models, linear, ridge, lasso and elastic regressions and their parameters, were used for model development. For each model, a grid search was used using leave-one-out cross validation (LOOCV) to optimize the model parameters. The scoring metric for the grid search was the negative mean absolute error (MAE). To ensure that the models were not unduly influenced by features with larger scales, the features were scaled on the training set before each fold in the LOOCV and then fitted onto the left-out dataset. The best model for each regression model was selected based on the LOOCV score. The selected models' performance was then evaluated on the unseen test dataset. The performance metrics calculated were the R2 scores, F-statistic, regression p value and the MAE. To provide a more robust estimate of the model's performance, bootstrapping was performed to calculate the 95th confidence intervals (Cls) for the R2 and MAE on the training data. This approach was applied to each feature set and a combination of all the features. The top performing model was selected for predicting PCPhenoAge and age acceleration using the performance metrics and model interpretability.

### Interpreting the Selected Model

Model interpretability was prioritized, aiming to understand the contributions of each feature to the model's predictions. To achieve this, KernelExplainer was used to calculate the Shapley additive explanations (SHAP) values, a unified measure of feature importance that allocates the contribution of each feature to the prediction for each sample.

### Weighted Gene Co-Expression Network Analysis (WGCNA) and Pathway Enrichment

WGCNA was performed with the WGCNA package in R software with default parameters for blockwise module construction. A weighted adjacency matrix was established by calculating Pearson correlations between each CpG site pair. The co-methylation similarity matrix was raised to a power f3=12 (R2 of 0.75) to calculate the weighted adjacency matrix. Values were used to construct a topological overlap matrix (TOM), which provided a similarity measure. The TOM was used to calculate the corresponding dissimilarity (1-TOM). Modules were created by applying average linkage hierarchical clustering to the TOM. The dynamic tree cutting algorithm (function=cutreeDynamic, deep split=0, minimum number of genes per module=250, cut height=0.15) was used to detect methylation modules. The assignment of outlying genes to modules was performed using the Partitioning Around Medoids (PAM) method. DNA methylation levels of CpG sites within a module were summarized with the module eigengene (ME) value, which is the overall methylation level of CpG sites clustering in a module. To identify relevant modules for the analysis, correlations between MEs and wearable features were calculated. Module membership (MM) was determined as the correlation of the ME and the individual CpG site. Module sites were determined by limiting the MM of individual sites to r>0.8. To assess the underlying biology of modules, CpG sites were annotated for genes using the Genome wide annotation for Human (org.Hs.eg.db) package39. Gene lists were input into EnrichR library40 for KEGG 2021 pathway enrichment. Significant pathways were selected using an adjusted p value<0.05. Gene lists were mapped for Entrez ID's and input into Pathview41 for visual analysis of the enriched pathways.

### Statistical Analysis

Daily means were calculated for all features, after which the 40-day mean was calculated from the daily means. For sleep features, the standard deviation and the coefficient of variance was calculated to quantify sleep consistency. Wherever correlations were performed the Pearson r and p value were calculated. The Benjamini-Hochberg false discovery rate (fdr) multiple hypothesis correction was applied top values. Displayed p values are corrected, unless stated otherwise, and statistical significance was set to p<0.05. Machine learning processes were performed using Python (version 3.9.0, Python Software Foundation). To further investigate the relationship of associations between age acceleration and wearable features, differentially methylated positions were calculated by using the ChAMP package, using age acceleration as the independent variable. The output set of DMPs were used for further downstream analysis.

### Results

Forty-six participants were included in the study. Age ranged from 24.8-75.5 years and the cohort were evenly distributed between males (n=25, 54%) and females (n=21, 46%). Participant characteristics are summarized in Table 1. Wearable-derived physiological, behavioural, and circadian features are summarized in Table 2. The feature set of sleep is very comprehensive (supplementary results). PCA was used to reduce the dimensionality of the sleep features. A threshold of 0.15 was implemented on the explained variance ratio, resulting in two components (explained variance ratio of 0.48 and 0.19 for PC1 and PC2 respectively). These components are representative of sleeping patterns used in further analysis. DNAm ages, in years, were calculated for study participants. The means and standard deviations (SD) for all ages were calculated for the study participants: chronological age (43.73±15.11), PCHorvath1 (54.3±12.0), PCHannum (57.2±12.7), PCGrimAge (55±12.8), PCPhenoAge (43.4±14.9). The range for the calculated DNAm age accelerations in years were PCHorvath1 (19.5), PCHannum (22.2), PCGrimAge (13.5), PCPhenoAge (25.9) and Phenotypic age (12.6).

TABLE 1
Study participant characteristics

| | Over 40 years (N = 21) | Under 40 years (N = 25) | |
|---|---|---|---|
| | Median [min, max] | Median [min, max] | p value |
| Chronological age (years) | 57.0 [40.0, 81.0] | 31.5 [24.0, 39.0] | <0.05 |
| Weight (kg) | 79.5 [53.0, 131] | 66.5 [51.0, 110] | <0.05 |
| Height (m) | 1.75 [1.53, 1.90] | 1.74 [1.59, 1.85] | 0.62 |
| BMI (kg/m²) | 26.0 [21.0, 42.0] | 22.0 [19.0, 34.0] | <0.05 |
| Systolic blood pressure (mmHg) | 122 [100, 165] | 110 [80.0, 147] | <0.05 |
| Diastolic blood pressure (mmHg) | 77.5 [62.0, 104] | 70.0 [56.0, 98.0] | 0.056 |

TABLE 2
Wearable features

| Feature | Description | Unit | Median (min max) | Feature set |
|---|---|---|---|---|
| VO2max | Maximal capacity of oxygen update and utilization by the | mL/kg/min | 36.94 [21 22, 60.54] | Physiological |
| | muscle | | | |
| Normalized crest | Indication of the contractile strength of the heart muscle. | Unitless | 0.29 [0.26, 0.33] | |
| time | The stronger the cardiac muscle, the faster the left ventricle | | | |
| | can eject stroke volume into the aorta | | | |
| Subendocardial | Indication of the amount of oxygenated blood volume or | Unitiess | 1.58 [0.68, 2.97] | |
| viability ratio | blood supply to the coronary arteries. A high SEVR indicates | | | |
| (SEVR) | good blood flow, whilst a low SEVR indicates poor blood flow | | | |
| Normalixed | Measurement of the resistance to blood flow in the small | Unitless | 0.63 [0.57, 0.69] | |
| pulse width/ | arteries SAR is related to arterial stiffness of the peripheral | | | |
| Small artery | arteries. Smaller SAR indicates healthier peripheral arteries | | | |
| resistance(SAR) | | | | |
| Large artery | Indication of the stiffness of the aorta and the wave travel | m/s | 1449 [805, 4117] | |
| stiffness index | speed in blood. Higher LASI is indicative of higher wave | | | |
| (LASI) | speed and, correspondingly, to higher artery stiffness | | | |
| B: A Ratio/Blood | Measurement of blood pressure. BPI value is proportianal to | Unitless | −0.55 [−0.64, −0.43] | |
| pressure index | average blood pressure | | | |
| (BPI) | | | | |
| Ejection duration/ | Representation of the blood pumping strength of the heart. A | Unitless | 0.4 [0.31, 0.53] | |
| Heart outflow | healthy HO value should not be too large or too small. | | | |
| (HO) | | | | |
| Resting heart | Frequency of heartbeats measured during state of rest | Bpm | 67 [42, 82] | |
| rate (RHR) | | | | |
| Weekly light | The amount of time, in hours, spent in light active state | Hours | 6.75 [0.45, 19.85] | Behavioral |
| active hours | over the duration of a week | | | |
| Weekly | The amount of time, in hours, spent in moderate active | Hours | 1.2 [0.1, 20.3] | |
| moderate active | state over the duration of a week | | | |
| hours | | | | |
| Weekly vigorous | The amount of time, in hours, spent in a vigorous active state | Hours | 0.5[0, 6.7] | |
| active hours | the duration of the week | | | |
| Daily sedentery | The amount of time, in hours, spent in a sedintary state over | Hours | 7.5 [4.7, 10.9] | |
| hours | thye duration of a day | | | |
| Sleep PC1 | The first principal component of sleep features | Arbitrary | −0.71 [−4.27, 7.71] | Circadian |
| Sleep PC2 | The second principal component of sleep features | Arbitrary | −0.12 [−3.11, 7.22] | |

Physiological features were correlated with behavioural and circadian features, as shown in FIG. 4. Behavioural and circadian features are plotted as rows, and physiological features as columns. Each cell represents the Pearson r value of the correlations on a sliding colour scale from negative (blue) to positive (red). Significance (p<0.05) is illustrated by a * next to the correlation value in the cell. Active minutes, in all intensity zones, and sedentary hours, were correlated with RHR, HO, SEVR, and VO2max. Light minutes, alone, was correlated with BPI. The first component of sleep was positively correlated with SEVR.

Correlations were calculated between wearable features and six different age calculations. After correcting for multiple hypothesis testing, correlations with PCPhenoAge and PCPhenoAge acceleration were identified (supplementary results S3). Through this result, PCPhenoAge and PCPhenoAge acceleration (referred to from here-on as age acceleration) were selected as targets for regression modelling. The feature set which elicited the highest accuracy across all models for predicting PCPhenoAge was all of the features, and for predicting age acceleration was the physiological feature set. The results of the four linear models used to predict PCPhenoAge and age acceleration are tabulated in table 3. The 5th and 95th confidence intervals (Cl) on model R2, during training, for all models was calculated through bootstrap resampling. For predicting both PCPhenoAge and age acceleration, in training, Linear regression performed the best out of the four models with the largest R2 value and lowest MAE in years. When the linear regression model was applied to test data the performance decreased for both metrics. When considering the performance of the regularized linear models, ridge regression performed the best on the training and test data (table 3), with higher R2, smaller training R2 Cls and lower MAE compared to the other models and showing similar performance between training and test data (FIG. 5A). The regularization of the ridge regression resulted in a tighter distribution of age acceleration predictions compared to actual age acceleration (FIG. 5B), predicting the lowest age acceleration as −5.6 years and the highest as 7.6 years. Based off these results, the ridge regression model was selected as the top performing model for predicting both PCPhenoAge and age acceleration.

To investigate the ridge regression model to predict age acceleration, SHAP values were used to quantify the individual contribution of features to the model which predicts age acceleration, shown in the unit of years (FIG. 5C), ranking the most impactful features from top to bottom. High SHAP values for RHR indicate that higher values of RHR contribute to a higher predicted age acceleration, suggesting that an increase in RHR generally leads to an increase in the model's output. Conversely, lower RHR values would lead to lower predicted age acceleration levels. The SHAP values for V02max indicate that higher V02max values lead to lower predicted age acceleration levels. Conversely, lower V02max values would lead to higher predicted age acceleration levels. LASI shows a similar pattern, but to a lesser extent to that of RHR, whereby higher values influence the model output to predict higher age acceleration. Other PWF features are shown to have a lesser impact on model predictions.

TABLE 3
Linear models to predict PCPhenoAge and PCPhenoAge acceleration

| Regression | | | | | | | | |
|---|---|---|---|---|---|---|---|---|
| Model | Training R2 | Test R2 | Training MAE | Test MAE | F - statistic | p value | Target | Feature set |
| Linear | 0.59 | 0.27 | 2.84 | 4.13 | 5.76 | 0.0003 | PCPhenoAge | Physiological |
| | [0.28-0.73] | | [2.25-3.46] | | | | Acceleration | |
| Ridge | 0.49 | 0. [symbol] 7 | 3.05 | 3.57 | [symbol] .86 | 0.005 | | |
| | [0.29-0.60] | | [2.38-3.84] | | | | | |
| Lasso | 0.35 | 0.34 | 3.34 | 3.75 | 2.21 | 0.06 | | |
| | [0.08-0.48] | | [2.44-4.33] | | | | | |
| Elastic Net | [symbol] | 0.32 | 3.31 | 3.76 | 2.2 | 0.0 [symbol] | | |
| | [0.17-0.46] | | [2.48-4.28] | | | | | |
| Linear | 0.71 | 0 | 6.18 | [symbol] .71 | 4.11 | 0.001 | PCPhenoAge | All |
| | [0.45-0.8 [symbol] ] | | [4.68-7.56] | | | | | |
| Ridge | 0.56 | 0.67 | 7.57 | 6.31 | 2.1 [symbol] | 0. [symbol] 5 | | All |
| | [0.27-0.7] | | [ [symbol] .83-9.52] | | | | | |
| Lasso | 0.53 | 0. [symbol] 3 | 7.85 | 7.16 | 1.8 [symbol] | 0.09 | | All |
| | [0.25-0. [symbol] ] | | [6.05-9.91] | | | | | |
| Elastic Net | 0.49 | 0.64 | 8.25 | 6.84 | 1.61 | 0.15 | | All |
| | [0.15-0.61] | | [6.2-10.2] | | | | | |

[symbol]

indicates data missing or illegible when filed

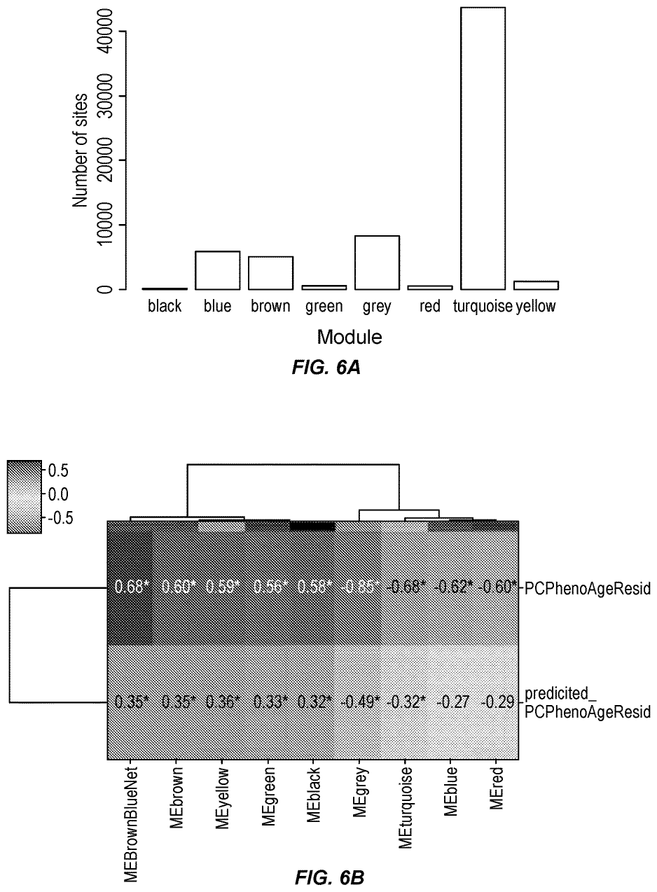

To further investigate the underlying biology of age acceleration and the connection to wearable data, normalized CpG site methylation levels were assessed for differentially methylated positions (DMP) with age acceleration as the predictor in a linear regression onto each site's methylation levels, using the ChAMP package. A total of 64 789 CpGsites were associated (adjusted p<=0.05) with age acceleration. This subset of CpG sites was used to construct the WGCNA modules. Seven modules were identified from the WGCNA (excluding the grey module). All modules had more than 250 CpG sites. The black module was the smallest of the modules with 251 CpG sites. The turquoise, blue, brown, and yellow modules were the largest modules and the 8850 uncorrelated CpG sites formed the grey module (FIG. 6A). The module eigengenes (ME) are the representative vector for the methylation status of the module for each participant. Correlations between MEs and the actual and the predicted age acceleration were computed. The MEs of all the modules was significantly correlated with age acceleration. Wearable predicted age acceleration was significantly correlated, to a lesser extent than actual age acceleration, to the MEs of all the modules (FIG. 6B).

Additionally, the wearable features which were used as input to the model to predict age acceleration were fit using a linear model onto the methylation status of each of age acceleration DMPs. The top-ranked sites were returned based on the adjusted p value associated with the F statistic of the regression. There were no significant findings using this selection criteria. An additional approach was taken whereby the R2 values for each regression of the DMPs was used to determine a cut-off value to identify CpG sites using the age acceleration model input features. Using the 5th percentile of the R2 values of the DMP regressions as a cutoff value, 5460 sites were selected, as hub sites, from the regressions of the model input features onto the CpG sites.

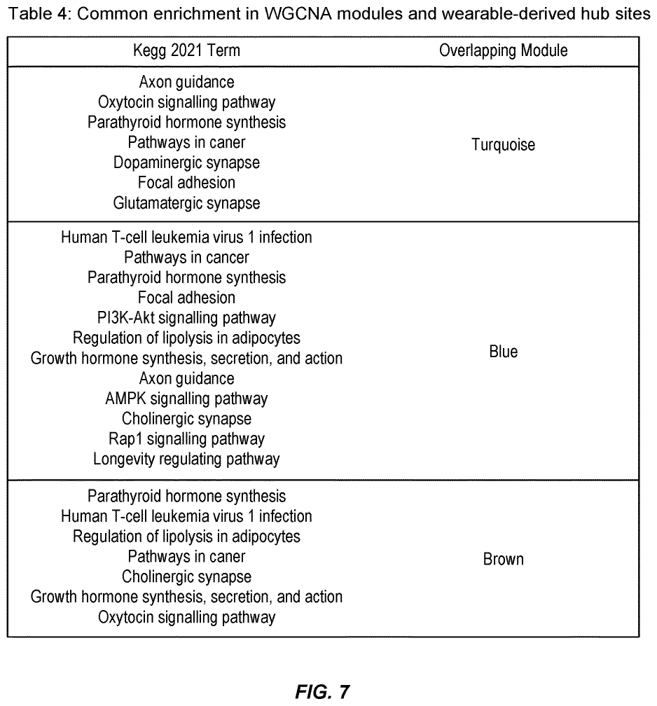

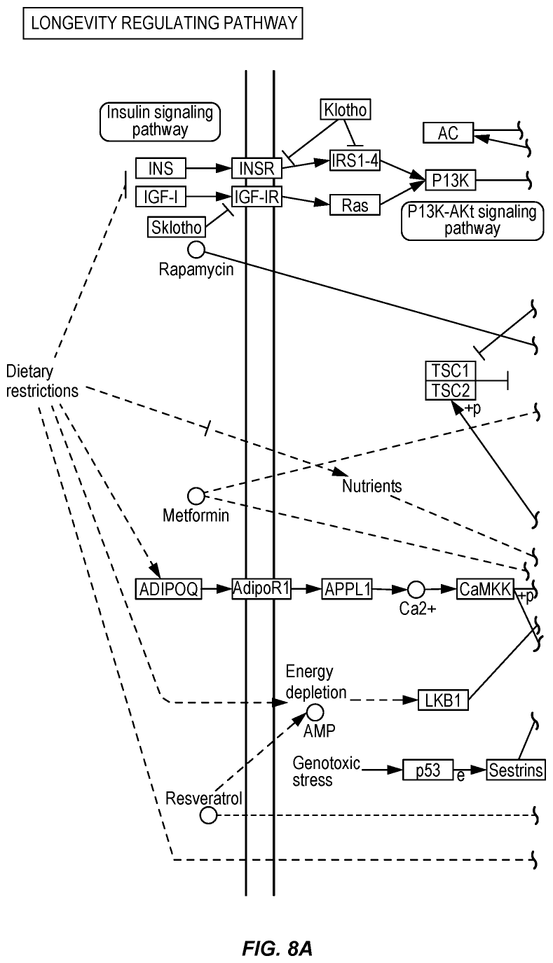

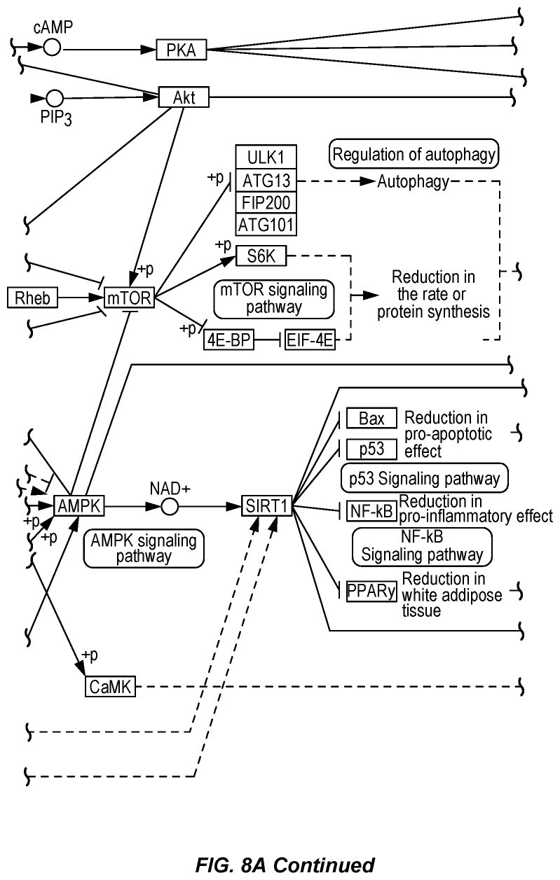

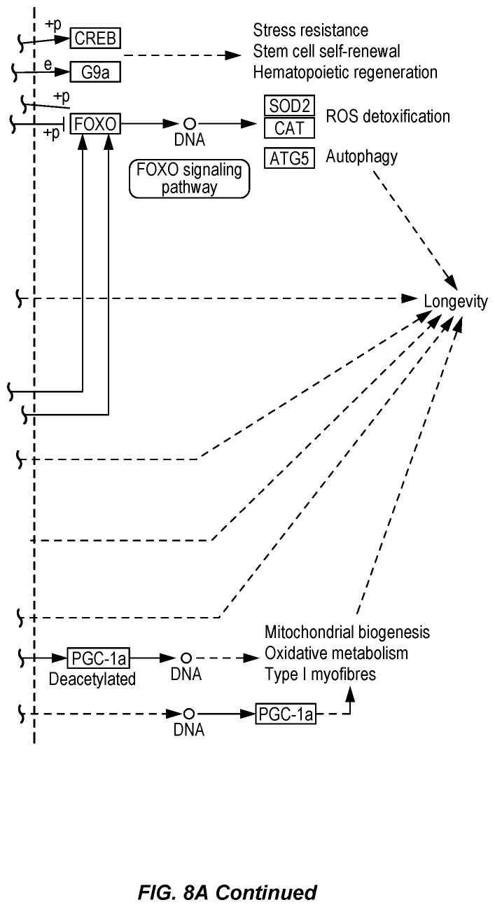

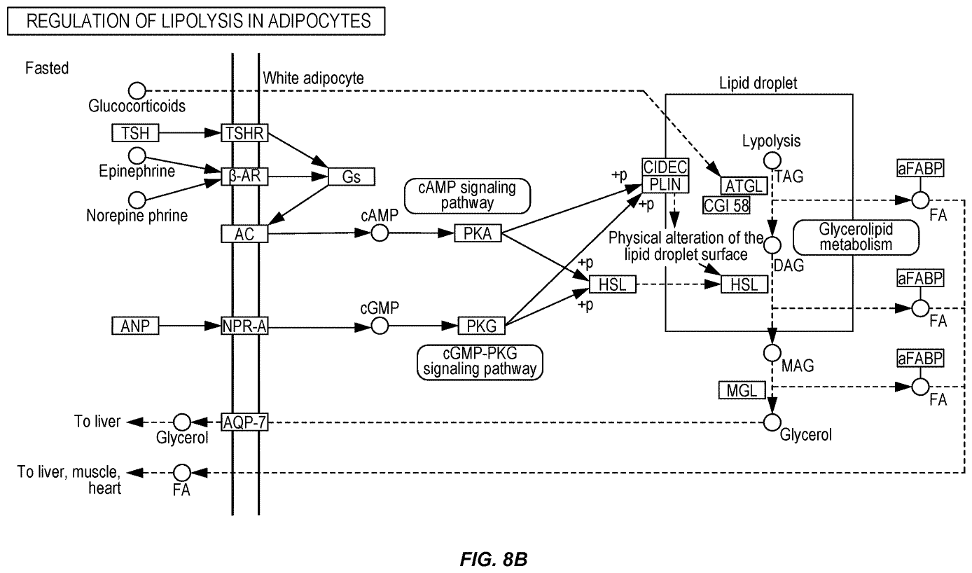

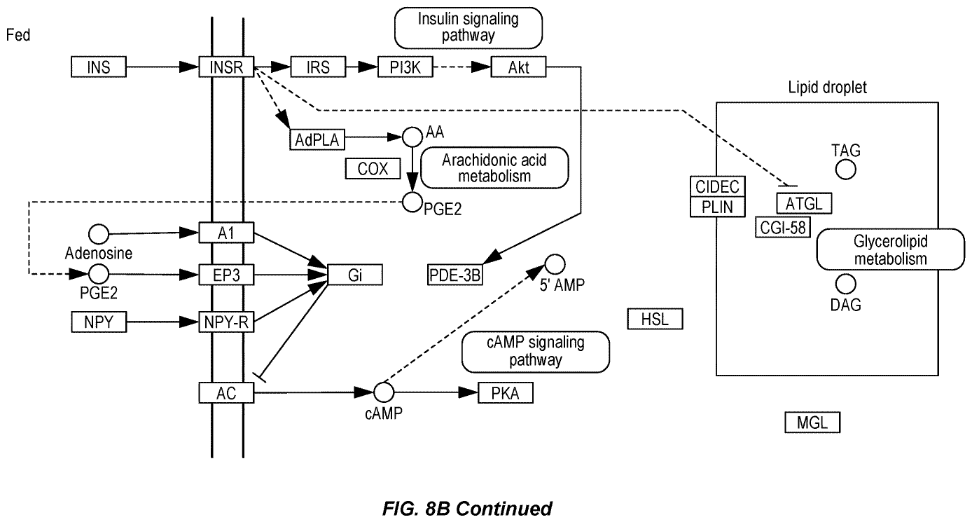

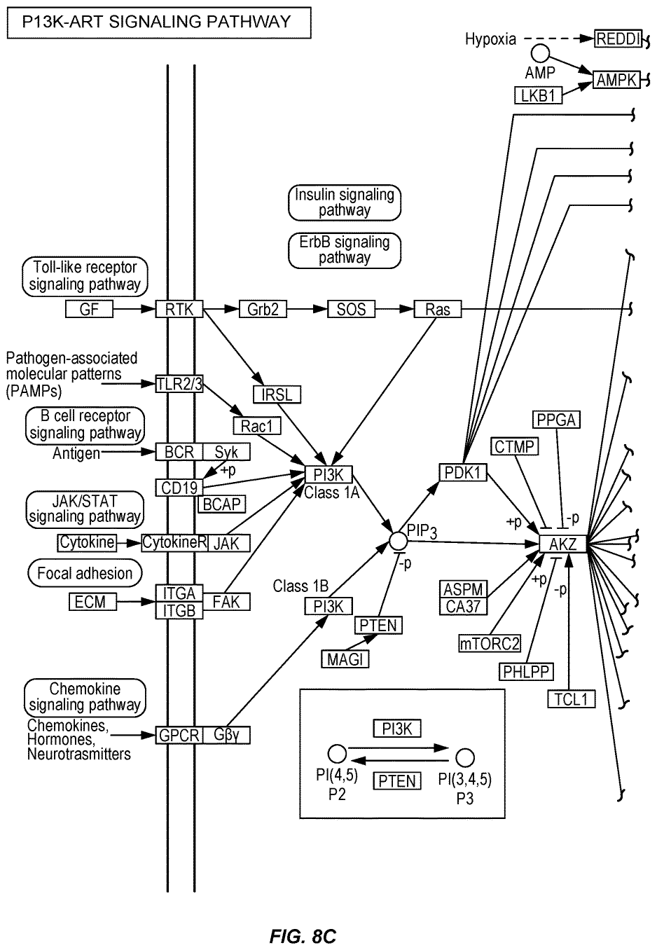

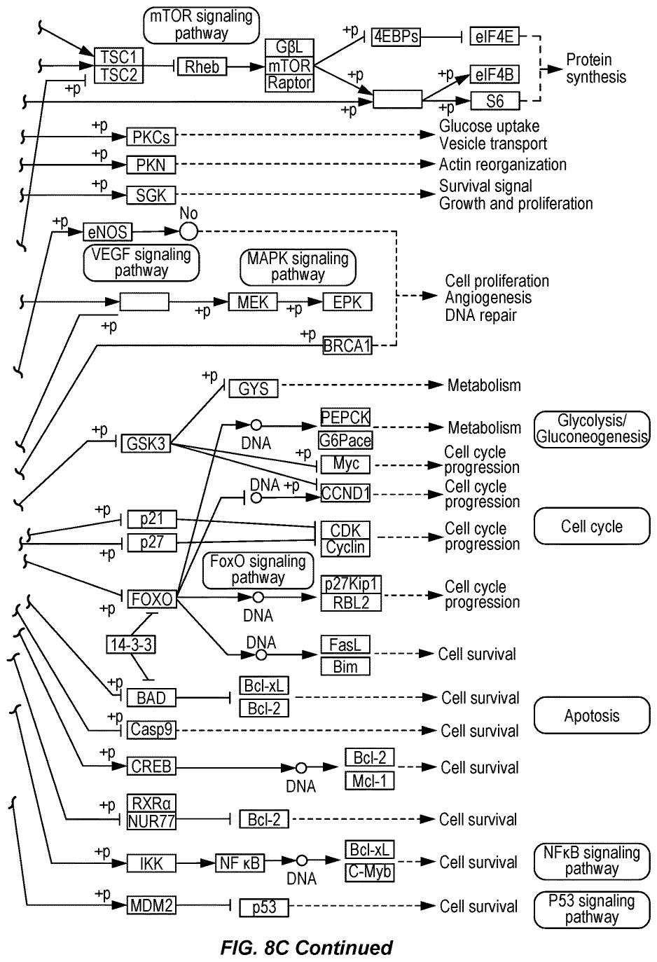

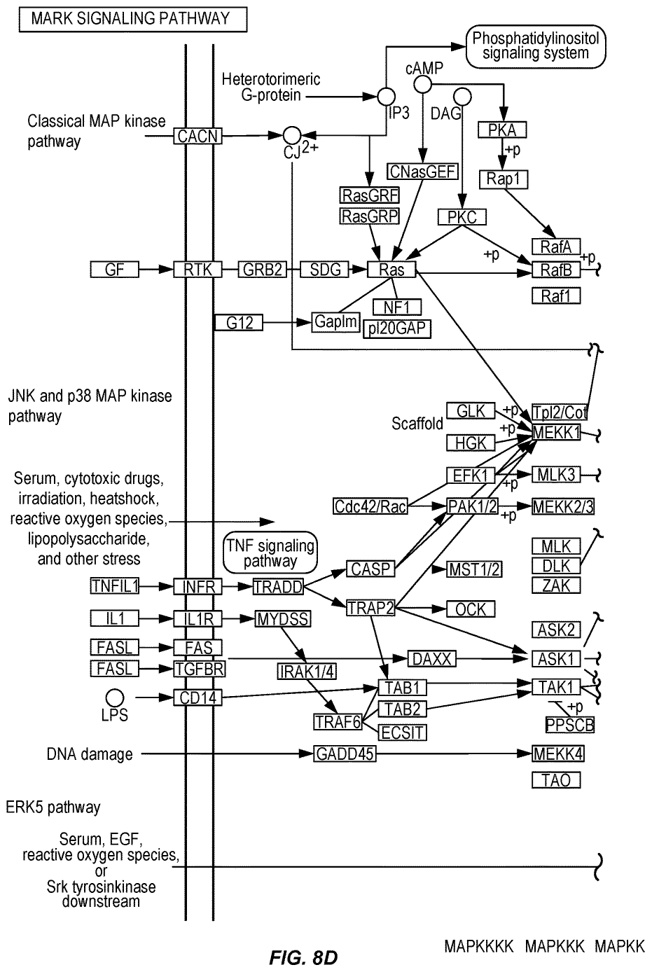

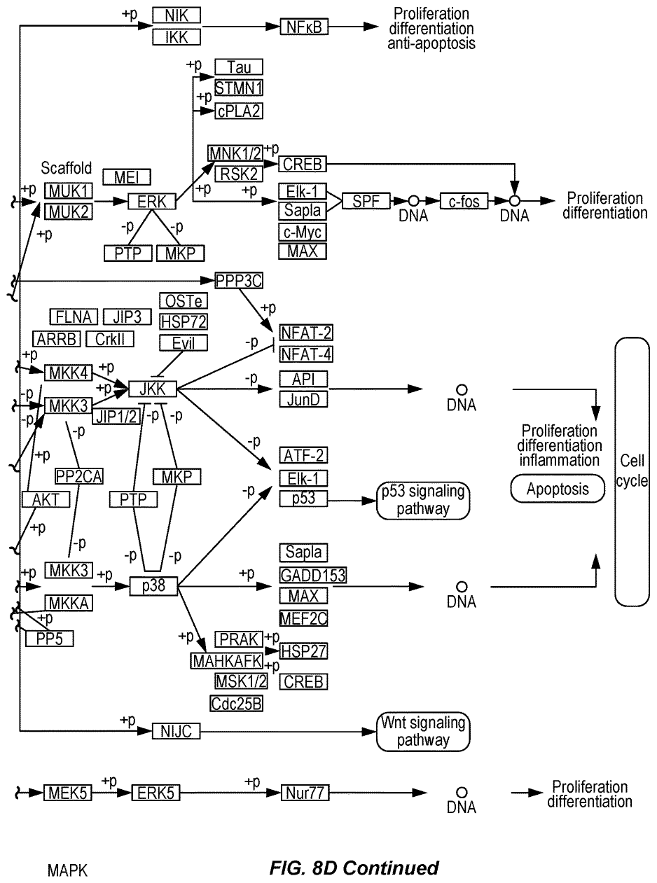

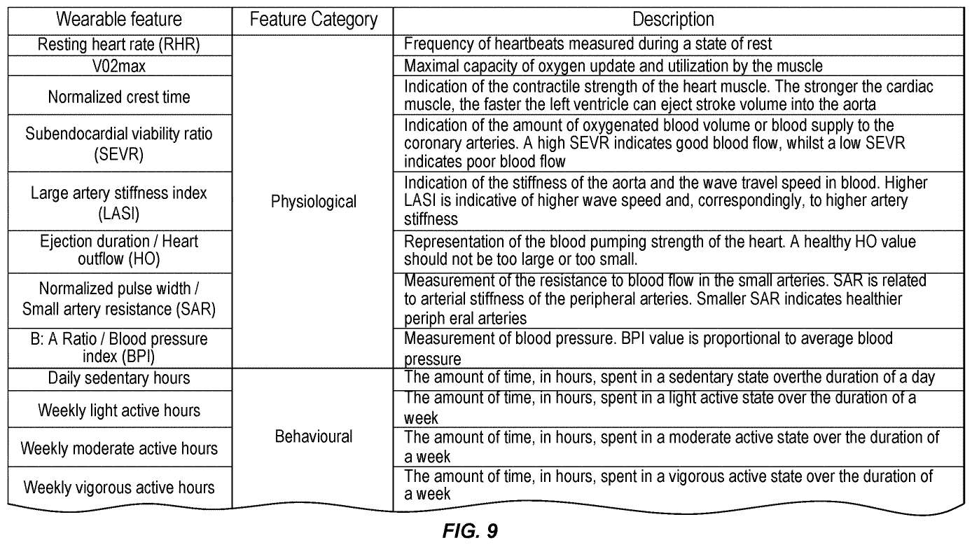

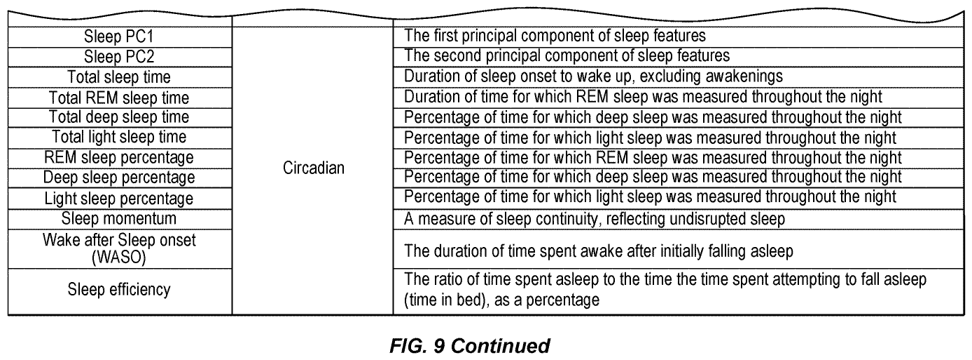

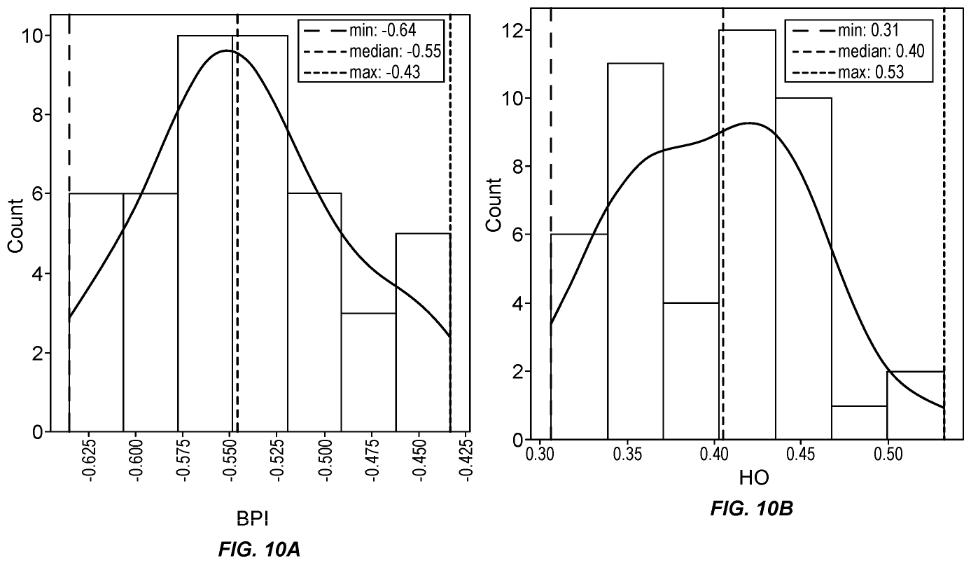

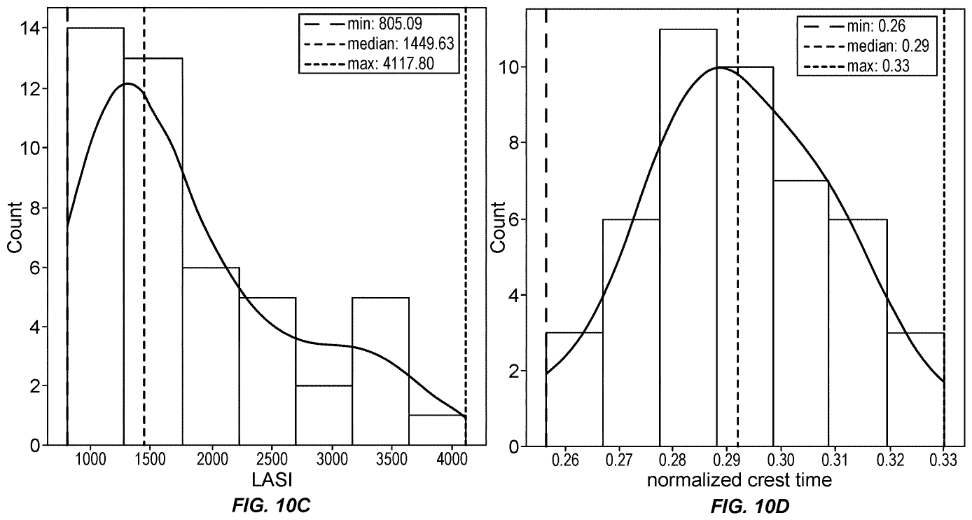

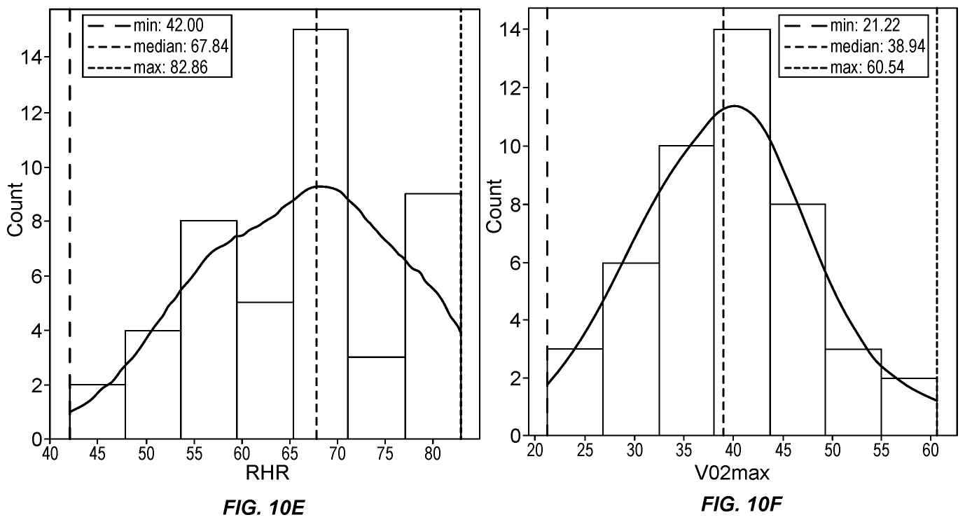

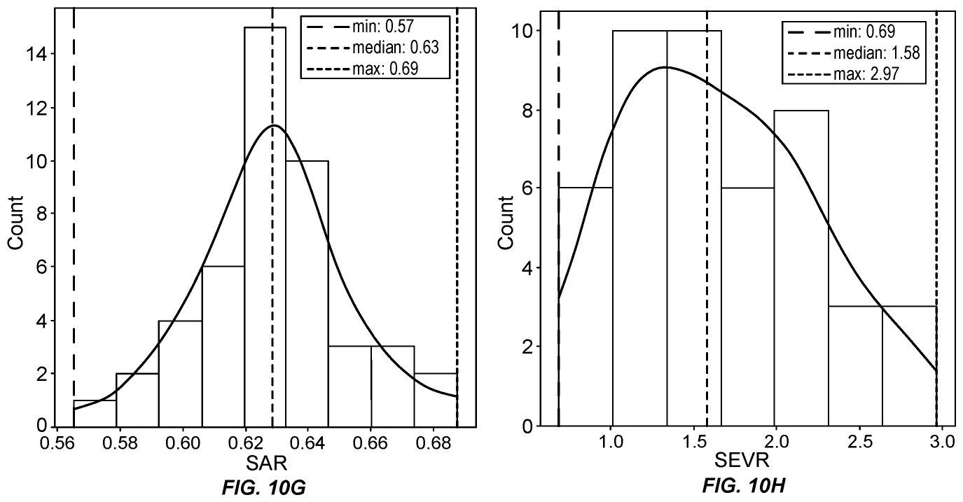

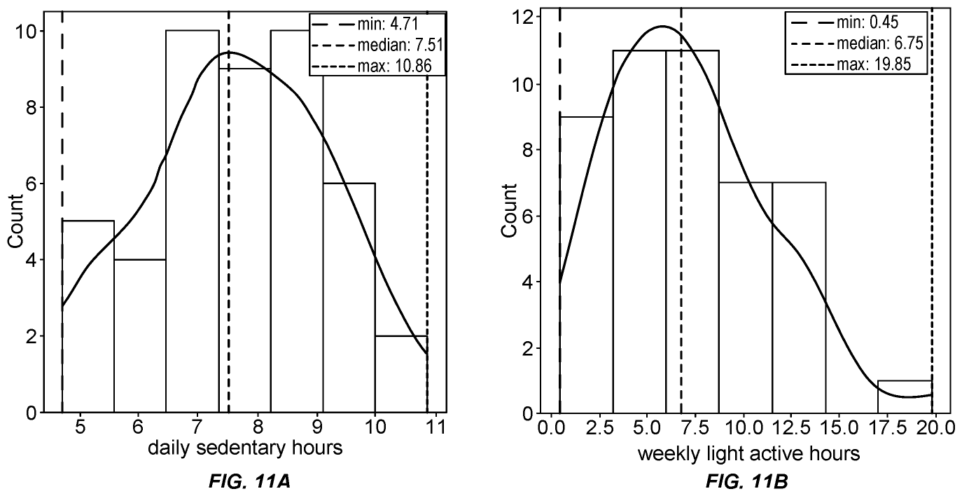

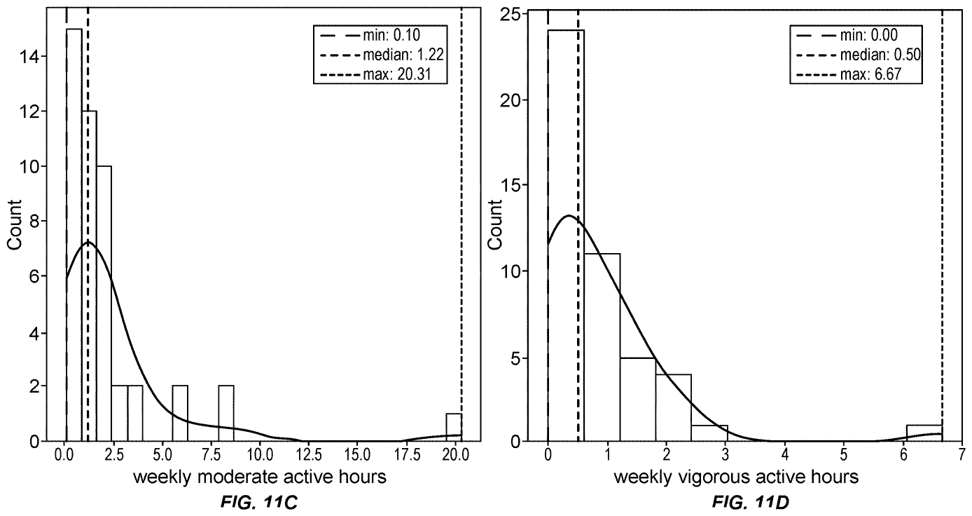

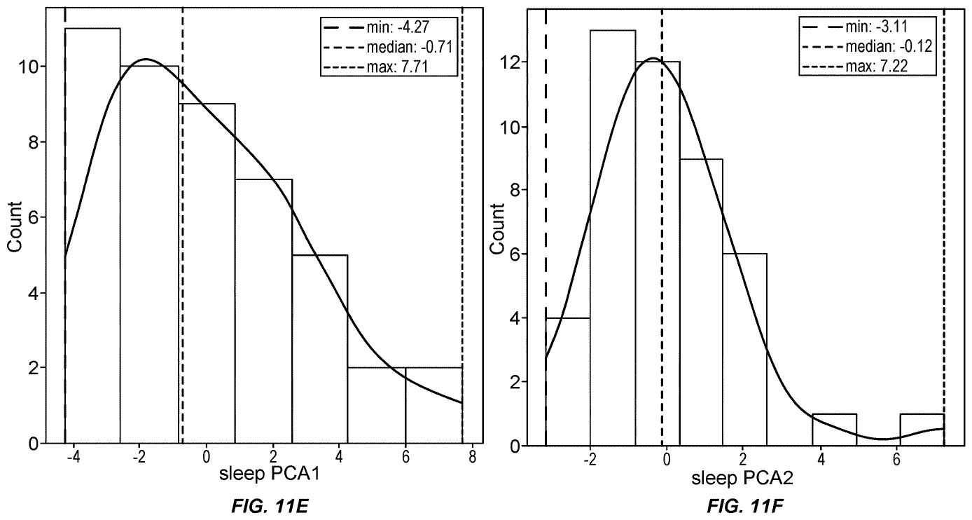

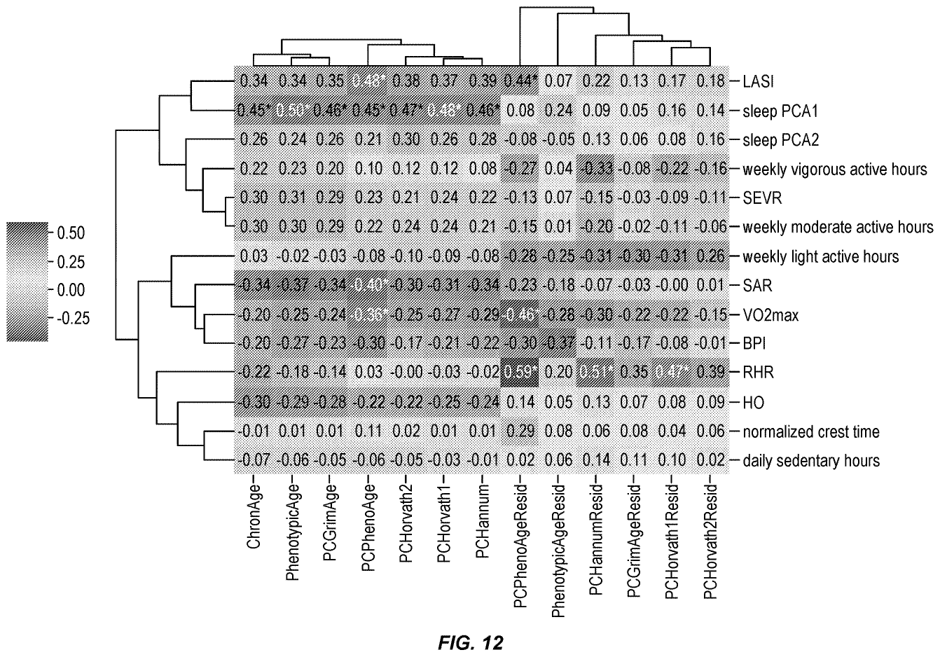

The wearable derived hub sites, and module sites from the WGCNA were annotated for their corresponding genes. Gene lists were input for pathway enrichment using the enrichR package (supplementary results S7). Considering the WGCNA modules, only the blue, brown, and turquoise modules were significantly enriched for pathways. There was an overlap of enriched pathways for the brown, blue and turquoise modules, and that of the wearable derived gene list (FIG. 7). There are pathways which are enriched the blue, brown, and turquoise module, such as axon guidance, parathyroid hormone synthesis and pathways in cancer. The brown and the blue modules seem to have a complementary relationship, as evidenced by the inverse relationship the ME had with wearable features, and the fact that both are enriched for similar pathways. Cell signalling pathways which are present in these modules are evident in many biologic al processes. From the enriched pathways, pathways of interest were selected for further investigation. The pathways selected for further analysis included cell signalling pathways such as Phosphoinositide 3-kinase (Pl3k)—Protein kinase B (Akt), and AMP-activated kinase (AMPK). These signalling pathways are involved in other enriched pathways, such as the longevity pathway and regulation of lipolysis in adipocytes, making them pathways of interest. The gene list and accompanying KEGG pathway were input into pathview and resulted in KEGG pathway images, whereby genes coloured in red are present in the wearable derived hub site annotated gene list (FIGS. 8A-8D).

Interestingly, the CpG sites for the pathways of interest for both the blue and the brown module were annotated to the same genes. Given the complementary effect of the blue and brown modules on the same pathways, we derived a measure to illustrate the over rail effect, The net ME is the arithmetic difference between the ME values of the blue and brown modules and gives a representation of the methylation status of the two modules as it pertains to the pathways of interest (supplementary figure). To investigate the relationship of this differential methylation with age acceleration, the net ME values were correlated to PCPhenoAge acceleration. Although the CpG sites for the blue and brown modules were annotated to the same genes, we found that the net effect of the methylation on the pathway genes differed between individuals, and that this effect strongly correlated with age acceleration (r=0.67, p=1.72e-07).

### Discussion

Physiological state is dynamic, sufficient stimulus needs to be placed on the organism to elicit adaptation. The present study illustrates an association with increased activity and decreased sedentary time and lower RHR and ejection duration index and higher VO2 max and SEVR. It is well established that lower RHR levels are associated with increased activity time. The mechanisms driving the change in RHR are complex and multifactorial but includes parasympathetic activation, decreased responsiveness to beta-adrenergic stimulation, decreased intrinsic heart rate or a combination of all of them. Whilst VO2 max has a genetic component to it, exercise training is shown to increase VO2 max levels. Peripheral and central adaptations, such as muscle perfusion, increased mitochondrial number and function, and increased stroke volume, occur due to increased activity. Structural changes to the heart and vasculature occur with various growth factors providing a cellular environment for myocardial remodelling. SEVR is a measure of myocardial perfusion, with higher values being indicative of high blood flow to the heart. Regular physical activity can improve cardiovascular health by increasing the supply of blood to the heart muscle. The association with sleep PCA1 and SEVR is not intuitive, as it illustrates an association between poor quality inconsistent sleep with high blood supply. The present study only captured wearable data for 40 days; making it difficult to characterize individual habits. A study design with a longer monitoring time would aid in capturing the more nuanced links between wearable derived features as a function of behaviour.

A complete measure of age acceleration is still not agreed upon in the aging field, the technique used in this study was the use of epigenetic clocks. One of the benefits of the use of these clocks is, by design, the output is in the scale of years. Other measures of age acceleration, such as population relativity data, are not as intuitive. Epigenetic age acceleration has been associated with an increase in ACM and disease onset. What is yet to be achieved is a long-term clinical trial with multiple time points and interventions associated with decreasing aging and ACM. A study of this stature will aid in validating the use of epigenetic clocks as a measure of biological age, In this paper a model utilizing wearable features to predict PCPhenoAge and age acceleration with moderate results was developed. This, to our knowledge, is the first study to link age acceleration with metrics derived from a PPG-based wearable, with other studies utilizing accelerometer-based wearable. Features derived from a combination of PPG and accelerometer data offer more biological insight to that of an accelerometer. The interpretation of the model with the use of the KernelExplainer revealed that the most impactful features, in the physiological feature set, were most notedly lower RHR, and higher LAS) and VO2 max predicting accelerated aging, followed by other PWF features. Increased arterial stiffness, measured by impedance cardiography, has been shown to associate with an increase in epigenetic age acceleration, validating the impact of LASI to our model. VO2max has been shown to negatively associate with a fitness specific DNAm clock, FitAge, but not PhenoAge acceleration. This speaks to a hypothesis that different epigenetic clocks capture different portions of aging. Although none of the behavioural features were included in the selected model, increased exercise and good sleeping habits have been shown to correlate with epigenetic age acceleration. A plausible explanation for this could be due to the small sample size (n=48) and the duration of the study being insufficient. A relationship between physical activity and sedentary time, measured by an accelerometer, and epigenetic age acceleration has been established by others.

WGCNA is an analysis developed by Horvath and Langtelder, originally for transcriptomic data, but has been used for multiple types of biological data including methylation data. The method clusters highly connected co-methylated CpG sites into a module, represented by a ME, a representation of the methylation status of the sites within the module, The predicted age acceleration correlates to the ME for each module to a lesser extent that actual age acceleration. Due to the regularization applied to the model, the predicted age acceleration has a smaller range (−5 to 6 years) in the population to that of actual age acceleration (−15 to 16 years). This condensed predicted age acceleration distribution decreases the strength of the association to MEs. Increasing the number of participants with age accelerations above and below 8 years in model training may result in more explained variance and a wider distribution of predicted age acceleration. The wearable features used as input to the age acceleration model were regressed onto each CpG site from the DMPs identified using PCPhenoAge acceleration. Although multiple hypothesis p value correction revealed no significant regressions, the approach was taken to identify CpG sites using the explained variance of the regression which mirrored that of the regression experiments conducted identifying the DMPs from the age acceleration regressions. The pathway enriched with genes associated of the 5460 sites identified with this technique overlapped with key aging pathways identified in the WGCNA module biology.

The turquoise module, being the largest, is difficult to summarize but can be classified as a module responsible for general cellular, and tissue maintenance module. The molecular footprint of aging includes general dysregulation of cellular and tissue homeostasis and maintenance pathways. A hallmark of aging is genetic instability. This will increase the chance of misfolding of proteins, for example. In a healthy cell, these proteins' would be recycled by the ubiquitin-mediated proteolysis and autophagy processes. If these processes are attenuated or the accumulation of proteins is above the capacity of autophagic flux these accumulated proteins can be detrimental, as seen progression of Alzheimer's disease. When referring to pathways from here on out, it specifically refers to the overlapping pathways which were enriched in each module and from the wearable derived CpG set. Pathways in cancer were enriched in the turquoise, blue and brown modules. The risk for cancer increases with age and epigenetic age acceleration. Molecular interactions which lead to cancers are complex and multi-factorial. Mutations to a key cancer gene, p53, have been noted in cancer patients. There is also an indirect impact on the cancer pathology whereby epigenetic modifications allow cancer cells to escape immune cell surveillance. There is evidence that increased physical activity reduces the risk of cancer and that up to 40% of the risk of cancer can be mitigated through lifestyle behaviours.

Many of the CpG sites for the blue and brown modules were annotated to the same genes, we found that the net effect of the methylation on the pathway genes differed between individuals, and that this effect strongly correlated with PCPhenoAge acceleration, allowing us to conclude that the specific methylation pattern in age-related pathways results in different aging phenotypes. Antiviral defence mechanisms have co-evolved with humans and include methylation of CpG sites to inactivate retrovirus expression. Metabolic risk factors such as elevated glucose, hypertension, increased BMI and insulin resistance are associated with epigenetic age acceleration13, and make up the metabolic syndrome. Lipolysis in adipocytes is the process which hydrolyses triglycerides (TAG) to release free fatty acids (FFA) and glycerol into circulation. The regulation of this process has 3 main signalling pathways, cAMP, guanosine 3′,5′-cyclic monophosphate (cGMP) and insulin signalling pathways, of which the genes involved in the latter are present in the wearable-derived gene list. The insulin signalling pathway involves binding of insulin to its receptor (INSR) initiating a cellular cascade activating Pl3K/Akt, ultimately inhibiting lipolysis. Dysregulation of lipolysis in adipocytes is seen in the aging phenotype, leading to lipid synthesis and ultimately increased adipose tissue as a structural change, but indirectly increases chronic inflammation that accompanies this dysregulation. Genes involved in the previously mentioned pathway as INSR/IRS/Pl3k/Akt occur in multiple cellular processes such as cellular metabolism and cell survival65, but of interest in this study is their enrichment in the longevity regulating pathway. Maintaining cellular homeostasis is paramount to cellular health and function. Multiple signalling pathways are responsible for this such as Pl3k/Akt/FoxO, Pl3k/Akt/mTOR (mechanistic target of rapamycin), which are present in the wearable derived gene list, and the Sirtuins. Pl3k/Akt/mTOR is a signalling pathway critical in regulating cellular growth and survival. mTOR can be activated by insulin and other growth signals. Under anabolic conditions, mTOR is responsible for protein and lipid synthesis, and the inhibition of autophagy. In low nutrient environments, mTOR is inhibited and autophagy is upregulated. Dysregulation of this process leads to an accumulation of damaged cells, a hallmark of aging.

The inclusion of the collection of genes in the wearable derived annotated gene list validates a connection between differential methylation of genes involved in molecular mechanisms of aging and physiological and structural state of humans captured by wearable devices. Our analysis highlights a new hypothesis, which is that by overcoming the study limitations listed below, it may be possible to associate changes in wearable derived behaviour to changes in the activity of individual cellular components, especially through continued epigenomic sampling combined with the use of a wearable device. For components with a known cellular function, such a finding can potentially advance cause-and-effect understanding of the impact of lifestyle on physiology and age acceleration, going beyond purely associative relationships. We note that investigation of the transcriptome to confirm predicted effects of methylation changes might in future help further extended findings towards causal mechanisms. Behavioural features impact physiological state, measured by the wearable. Wearable derived physiological features give an indication of health that is more strongly correlated with biological age than chronological age and correlated with age acceleration. These physiological features are a by-product of behavioural features, for example, increased activity time and decreased sedentary time are linked to improved physiological metrics. Exploration of the underlying biology of age acceleration presented trend correlations with behavioural and physiological features and methylation of genes in pathways relating to the aging process. This study presents the case that PPG-based wearable devices can capture portions of biological aging and should be used as a tool, in combination with other measurement techniques, for monitoring and potentially, in future, helping to manage healthy aging.

The current study, although novel, has its limitations. Human research is accompanied by natural variation amongst the participants of the study cohort. Increasing the sample size aids in limiting the variation observed. A limitation to this study was the small sample size, when comparing analysis techniques used by other studies. The study design was cross-sectional, only allowing for associations to be made between metrics measured. A longitudinal study with multiple measures of epigenetic age will provide an opportunity for assessing the changes in biological age over time and how the wearable is able to track those changes. Additionally, it will be interesting to assess to what degree wearable features respond to interventions which are known to slow down the rate of aging. This study did not target the recruitment of individuals with compromised health. Adding these individuals to future studies would make the observed associations more robust and allow the analysis to group the participants, allowing causal relationships to be discovered.

Although several aspects have been disclosed in the foregoing specification, it is understood by those skilled in the art that many modifications and other aspects will come to mind to which this disclosure pertains, having the benefit of the teaching presented in the foregoing description and associated drawings. It is thus understood that the disclosure is not limited to the specific aspects disclosed hereinabove, and that many modifications and other aspects are intended to be included within the scope of any claims that can recite the disclosed subject matter.

It should be emphasized that the above-described aspects are merely possible examples of implementations, merely set forth for a clear understanding of the principles of the present disclosure. Many variations and modifications can be made to the above-described aspect(s) without departing substantially from the spirit and principles of the present disclosure. Further, the scope of the present disclosure is intended to cover any and all combinations and sub-combinations of all elements, features, and aspects discussed above. All such modifications and variations are intended to be included herein within the scope of the present disclosure, and all possible claims to individual aspects or combinations of elements or steps are intended to be supported by the present disclosure.

The complete disclosure of all patents, patent applications, and publications, and electronically available material cited herein and/or listed below are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.
Awad, Aymen A., Ala S. Haddadin, Hossam Tantawy, Tarek M. Badr, Robert G. Stout, David G. Silverman, and Kirk H. Shelley. “The relationship between the photoplethysmographic waveform and systemic vascular resistance.” Journal of clinical monitoring and computing 21, no. 6 (2007): 365-372.
Birrer, V., Elgendi, M., Lambercy, O. and Menon, C., 2024. Evaluating reliability in wearable devices for sleep staging. npj Digital Medicine, 7(1), p. 74.
Gil, E., Orini, M., Bailón, R., Vergara, J. M., Mainardi, L. & Laguna, P., 2010. Photoplethysmography pulse rate variability as a surrogate measurement of heart rate variability during non-stationary conditions. Physiological Measurement, 31(9), pp. 1271-1290.
Horvath, S. (2013). DNA methylation age of human tissues and cell types. Genome Biology, 14(10), R115.
Huang, N. and Selvaraj, N., 2020, July. Robust ppg-based ambulatory heart rate tracking algorithm. In 2020 42nd Annual International Conference of the IEEE Engineering in Medicine & Biology Society (EMBC) (pp. 5929-5934). IEEE.
Konopka, A. R., Laurin, J. L., Schoenberg, H. M., Reid, J. J., Castor, W. M., Wolff, C. A., Musci, R. V., Safairad, O.D., Linden, M. A., Biela, L. M. and Bailey, S. M., 2019. Metformin inhibits mitochondrial adaptations to aerobic exercise training in older adults. Aging cell, 18(1), p.e12880.
Levine, ME., Lu, A. T., Quach, A., Chen, B. H., Assimes, T. L., Bandinelli, S., Hou, L., Baccarelli, A. A., Stewart, J. D., Li, Y. and Whitsel, E. A., 2018. An epigenetic biomarker of aging for lifespan and healthspan. Aging (albany NY), 10(4), p. 573.
Muggeridge, D. J., Hickson, K., Davies, A. V., Giggins, O. M., Megson, I. L., Gorely, T. and Crabtree, D. R., 2021. Measurement of heart rate using the polar OH1 and Fitbit charge 3 wearable devices in healthy adults during light, moderate, vigorous, and sprint-based exercise: validation study. JMIR mHealth and uHealth, 9(3), p.e25313.
Laurence Richard Olivier, Franco Du Preez, Wikus Theo Villet, Janina De Wit, Melissa Kistner, Tom Delaubenfels, Etienne Terblanche, “Detection and treatment of ectopic pregnancy,” World Intellectual Property Organization, International Publication Number WO 2022147271 A1, published Jul. 7, 2022, filed Dec. 30, 2021, Patent Cooperation Treaty Application PCT/US2021/065709.
Sugden, C., du Preez, F. B., Olivier, L. R. and Deffur, A., 2023. Wearable-ome meets epigenome: A novel approach to measuring biological age with wearable devices. bioRxiv, pp. 2023-04.
Takazawa, Kenji, Nobuhiro Tanaka, Masami Fujita, Osamu Matsuoka, Tokuyu Saiki, Masaru Aikawa, Sinobu Tamura, and Chiharu Ibukiyama. “Assessment of vasoactive agents and vascular aging by the second derivative of photoplethysmogram waveform.” Hypertension 32, no. 2 (1998): 365-370.
Yang, D., Zhu, J. and Zhu, P., 2015. SpO2 and heart rate measurement with wearable watch based on PPG.
Aunan J R, Watson M M, Hagland H R, S0reide K. Molecular and biological hallmarks of ageing. British Journal of Surgery 2016; 103: e29-46.
Kaeberlein M. Longevity and aging. F1DOOPrime Rep 2013; 5: 1-8.
Petsko G A. A seat at the table. Genome Biol 2008; 9: 113.
Seals D R, Justice I N, Larocca T J. Physiological geroscience: Targeting function to increase healthspan and achieve optimal longevity. Journal of Physiology 2016; 594: 2001-24.
Lopez-Otfn C, Blasco M A, Partridge L, Serrano M, Kroemer G. The Hallmarks of Aging Europe PMC Funders Group. Cell 2013; 153: 1194-217.
Jones M J, Goodman S J, Kobor M S. DNA methylation and healthy human aging. Aging Cell 2015; 14: 924-32.
Seale K, Horvath S, Teschendorff A, Eynon N, Voisin S. Making sense of the ageing methylome. Nat Rev Genet 2022; 23: 585-605.
Horvath S. DNA methylation age of human tissues and cell types. Genome Biol 2013; 14. DOI:10.1186/gb-2013-14-10-r115.
Hannum G, Guinney J, Zhao L, et al. Genome-wide Methylation Profiles Reveal Quantitative Views of Human Aging Rates. Mol Cell 2013; 49: 359-67.
Lu A T, Quach A, Wilson J G, et al. DNA methylation GrimAge strongly predicts lifespan and healthspan. Aging 2019; 11: 303-27.
Levine M E, Lu A T, Quach A, et al. An epigenetic biomarker of aging for lifespan and healthspan. bioRxiv 2018; 10: 573-91.
Rezwan Fl, Imboden M, Amaral A F S, et al. Association of adult lung function with accelerated biological aging. Aging 2020; 12: 518-42.
Ammous F, Zhao W, Ratliff S M, et al. Epigenetic age acceleration is associated with cardiometabolic risk factors and clinical cardiovascular disease risk scores in African Americans. Clin Epigenetics 2021; 13: 1-13.
Perna L, Zhang Y, Mons U, Holleczek B, Saum K U, Brenner H. Epigenetic age acceleration predicts cancer, cardiovascular, and all-cause mortality in a German case cohort. Clin Epigenetics 2016; 8: 1-7.
Liu D, Aziz N A, Pehlivan G, Breteler M M B. Cardiovascular correlates of epigenetic aging across the adult lifespan: a population-based study. Geroscience 2023. DOl:10.1007/s11357-022-00714-0.
Ritz B R, Horvath S. Increased epigenetic age and granulocyte counts in the blood of Parkinson's disease patients. Aging 2015; 7: 1130-42.
Pellegrini C, Pirazzini C, Sala C, et al. A Meta-Analysis of Brain DNA Methylation Across Sex, Age, and Alzheimer's Disease Points for Accelerated Epigenetic Aging in Neurodegeneration. Front Aging Neurosci 2021; 13: 1-21.
Durso D F, Bacalini M G, Sala C, et al. Acceleration of leukocytes' epigenetic age as an early tumor and sex-specific marker of breast and colorectal cancer. Oncotarget 2017; 8: 23237-45.
Okazaki S, Numata S, Otsuka I, et al. Decelerated epigenetic aging associated with mood stabilizers in the blood of patients with bipolar disorder. Transl Psychiatry 2020; 10. DOl:10.1038/s41398-020-0813-y.
Katrinli S, Stevens J, Wani A H, et al. Evaluating the impact of trauma and PTSD on epigenetic prediction of lifespan and neural integrity. Neuropsychopharmacology 2020; 45: 1609-16.
Quach A, Levine M E, Tanaka T, et al. Epigenetic clock analysis of diet, exercise, education, and lifestyle factors. Agro Food Ind Hi Tech 2018; 29: 20-1.
Li X, Joehanes R, Hoeschele I, et al. Association between sleep disordered breathing and epigenetic age acceleration: Evidence from the Multi-Ethnic Study of Atherosclerosis. EBioMedicine 2019; 50: 387-94.
Carskadon M A, Chappell K R, Barker D H, et al. A pilot prospective study of sleep patterns and DNA methylation-characterized epigenetic aging in young adults. BMC Res Notes 2019; 12: 583.
Sugden K, Hannon E J, Arseneault L, et al. Patterns of Reliability: Assessing the Reproducibility and Integrity of DNA Methylation Measurement. Patterns 2020; 1: 100014.
Higgins-chen AT, Thrush K L, Wang Y, et al. clocks: Implications for clinical trials and longitudinal tracking. 2022; 2: 644-61.
Griffin P. Ultra-cheap and scalable epigenetic age predictions with TIME-Seq. 2021. 001:10.1101/2021.10.25.465725.
Iconaru El, Ciucurel M M, Georgescu L, Ciucurel C. Hand grip strength as a physical biomarker of aging from the perspective of a Fibonacci mathematical modeling. BMC Geriatr 2018; 18: 1-9.
Betik A C, Hepple R T. Determinants of V02 max decline with aging: An integrated perspective. Applied Physiology, Nutrition and Metabolism 2008; 33: 130-40. McGreevy K M, Radak Z, Torma F, et al. DNAmFitAge: biological age indicator incorporating physical fitness. Aging 2023; 15: 1-35.
McEniery C M, Yasmin, Hall I R, Qasem A, Wilkinson I B, Cockcroft J R. Normal vascular aging: Differential effects on wave reflection and aortic pulse wave velocity—The Anglo-Cardiff Collaborative Trial (ACCT). J Am Coll Cardiol 2005; 46: 1753-60.
Yang J, Yang L, Liu W, et al. Analysis of the Radial Pulse Wave and its Clinical Applications: A Survey. IEEE Access 2021; 9: 157940-59.
Gartstein. Photoplethysmography Sensors and Their Potential Future Applications in Health Care. Physiol Behav 2016; 176: 139-48.
Allen J. Photoplethysmography and its application in clinical physiological measurement. Physiol Meas 2007; 28. 001:10.1088/0967-3334/28/3/R01.
Matthews C E, Hagstromer M, Pober O M, Bowles H R. Best Practices for Using Physical Activity Monitors. Med Sci Sports Exerc 2012; 44: 1-17.
McIntyre R L, Rahman M, Vanapalli S A, Houtkooper R H, Janssens G E. Biological Age Prediction From Wearable Device Movement Data Identifies Nutritional and Pharmacological Interventions for Healthy Aging. Frontiers in Aging 2021; 2: 1-11.
R Core. R: A language and environment for statistical computing. R Foundation for Statistical Computing, Vienna, Austria. URL https://www.R-project.org/. 2021.
Tian Y, Morris T, Stirling L, Feber A, Teschendorff A. Chip Analysis Methylation Pipeline for Illumina HumanMethylation450 and EPIC. 2019.
Zhang B, Horvath S. A general framework for weighted gene co-expression network analysis. Stat Appl Genet Mol Biol 2005; 4. DOI:10.2202/1544-6115.1128.
Carlson M. org.Hs.eg.db: Genome wide annotation for Human. 2022.
Jawaid W. enrichR. 2022. https://maayanlab.cloud/Enrichr/.
Luo W. Pathview: a tool set for pathway based data integration and visualization. 2023. https://github.com/data pplab/pathview.
Hedayatpour N, Falla D. Physiological and Neural Adaptations to Eccentric Exercise: Mechanisms and Considerations for Training. Biomed Res Int 2015; 2015. 001:10.1155/2015/193741.
Reimers A K, Knapp G, Reimers C D. Effects of exercise on the resting heart rate: A systematic review and meta-analysis of interventional studies. J Clin Med 2018; 7. DOl:10.3390/jcm7120503.
Matsui K, Sugano S. Relation of intrinsic heart rate and autonomic nervous tone to resting heart rate in the young and the adult of various domestic animals. Nippon juigaku zasshi The Japanese journal of veterinary science 1989; 51: 29-34.
Stratton J R. Exercise Training Bradycardia Is Largely Explained. Int J Cardiol 2017; 213-6.
Tangen E M, Gjestvang C, Stensrud T, Haakstad L A H. Is there an association between total physical activity level and VO2max among fitness club members? A cross-sectional study. BMC Sports Sci Med Rehabil 2022; 14: 1-8.
Qiu Y, Pan X, Chen Y, Xiao J. Hallmarks of exercised heart. J Mal Cell Cardiol 2022; 164: 126-35.
Tang S, Huang W, Wang S, et al. Effects of aquatic high-intensity interval training and moderate-intensity continuous training on central hemodynamic parameters, endothelial function and aerobic fitness in inactive adults. J Exerc Sci Fit 2022; 20: 256-62.
Matyas Jokai, Ferenc Torma, Kristen M. McGreevy, Erika Koltai, Zoltan Bori, Gergely Babszki, Peter Bakonyi, Zoltan Gombos, Bernadett Gyorgy, Dora Aczel, Laszlo Toth, Peter Osvath, Marcell Fridvalszky, Timea Teglas, Balazs Ligeti, Regina Kalcsevszki, Aniko Z R. DNA methylation clock DNAmFitAge shows regular exercise is associated with slower aging and systemic adaptation. medRxiv 2022; 27: 58-66.
Spartano N L, Wang R, Yang Q, et al. Association of Accelerometer-Measured Physical Activity and Sedentary Time with Epigenetic Markers of Aging. Med Sci Sports Exerc 2023; 55: 264-72.
Kresovich J K, Garval E L, Martinez Lopez A M, et al. Associations of Body Composition and Physical Activity Level With Multiple Measures of Epigenetic Age Acceleration. Am J Epidemiol 2021; 190: 984-93.
Carroll J E, Ross K M, Horvath S, et al. Postpartum sleep loss and accelerated epigenetic aging. Sleep Health 2021; 7: 362-7.
Langfelder P, Horvath S. WGCNA: An R package for weighted correlation network analysis. BMC Bioinformatics 2008; 9. DOl:10.1186/1471-2105-9-559.
Tian Z, He W, Tang J, et al. Identification of important modules and biomarkers in breast cancer based on WGCNA. Onco Targets Ther 2020; 13: 6805-17.
Pei G, Chen L, Zhang W. WGCNA Application to Proteomic and Metabolomic Data Analysis, 1st edn. Elsevier Inc., 2017 DOl:10.1016/bs.mie.2016.09.016.
Tremblay B L, Guenard F, Lamarche B, Perusse L, Vohl M C. Network analysis of the potential role of DNA methylation in the relationship between plasma carotenoids and lipid profile. Nutrients 2019; 11. DOl:10.3390/nu11061265.
Hartl F U. Cellular Homeostasis and Aging. Annu Rev Biochem 2016; 85: 1-4.
Pohl C, Dikic I. Cellular quality control by the ubiquitin-proteasome system and autophagy. Science (1979) 2019; 366: 818-22.
Watanabe Y, Taguchi K, Tanaka M. Ubiquitin, Autophagy and Neurodegenerative Diseases. Cells 2020; 9: 1-15.
Hu J, Cao J, Topatana W, et al. Targeting mutant p53 for cancer therapy: direct and indirect strategies. J Hematol Oneal 2021; 14: 1-19.
Cao J, Yan Q. Cancer Epigenetics, Tumor Immunity, and immunotherapy. Trends Cancer 2020; 6:580-92.
Friedenreich C M, Ryder-Burbidge C, McNeil J. Physical activity, obesity and sedentary behavior in cancer etiology: epidemiologic evidence and biologic mechanisms. Mot Oneal 2021; 15: 790-800.
Huang P L. A comprehensive definition for metabolic syndrome. DMM Disease Models and Mechanisms 2009; 2: 231-7.
Ahmadian, Maryam (Department of Nutritional Science and Toxicology, University of California, Berkeley, California 94720) Wang, Yuhui (Department of Nutritional Science and Toxicology, University of California, Berkeley, California 94720), Sul, Hei Sook C 94720). Medicine in Focus: Lipolysis in Adipocytes. Int J Biochem Cell Biol 2010; 42: 555-9.
Hemmings B A, Restuccia D F. Pl3K-PKB/Akt Pathway. Cold Spring Harb Perspect Med 2012; 4:1-4.
Yao, Liping, and Weizhang Liu. “The hypertension assessment based on features extraction using PPG signal and its derivatives.” Physiological Measurement (2020).
Zhang, Yahui, Zhihao Jiang, Lin Qi, Lisheng Xu, Xingguo Sun, Xinmei Chu, Yanling Liu, Tianjing Zhang, and Stephen E. Greenwald. “Evaluation of cardiorespiratory function during cardiopulmonary exercise testing in untreated hypertensive and healthy subjects.” Frontiers in physiology 9 (2018): 1590.

What is claimed:

1. A system comprising:
a. At least one wearable device that collects raw sensor data that
i. tracks cardiovascular activity; and
ii. tracks motion
b. a computing device with memory, communication means, and processors, wherein the processors are configured to:
i. that perform digital signal processing of the raw wearable data via a series of digital signal processing algorithms; and
ii. extracts wearable derived physiological biometrics (WDPB) from the results of said digital signal processing algorithms using a series of A series of extraction algorithms;
iii. extract wearable derived behavioral biometrics (WDBB) from the results of said digital signal processing algorithms; and
iv. utilize WBAPM for predicting biological age and biological age acceleration, where these models use physiological and behavioral biometrics (WDPB and WDBB) as input and have been trained/are iteratively trained on the output models that predict biological age and biological age acceleration from HBD by application of the BAF principal; and
c. a user interface located on a wrist-worn wearable, a mobile device, a laptop, or a desktop display to display the biological age and biological age acceleration.

2. The system of claim 1 where predictions of biological are averaged over several days in order to reduce the degree of day over day variability seen in BAF based models with low frequency sampling and hence make it possible to perform trend analysis on a shorter timescale.

3. The system of claim 2, wherein the system is configured to provide the predictions daily, in order to provide more frequent estimates of biological age from wearable data than what is feasible by doing repeated sample collection and HBD production for BAF-based biological age prediction.

4. The system of claim 2 wherein additional behavioral biometrics (ABB) are recorded from other applicable IOT device(s) capable of transmitting lifestyle behavioral information and additional physiological biometrics (APB) are recorded from other applicable IOT device(s) capable of transmitting lifestyle behavioral information.

5. The system of claim 4, wherein Behavior-Physiology Temporal Causal Analysis (BPTCA) is used to discover statistically significant relationships between QCB and subsequent changes in biological age as predicted from the wearable device data, with a time delay ranging from zero days to one month.

6. The system of claim 5, wherein candidate causal relationships (CCRs) between voluntary behavior and wearable-derived biological age are discovered on the data of a population of users and used to extrapolate insights from the larger population, to the behavior recorded for a single user, wherein projected consequences of a user's behavior can be communicated to the user to provide a strong feedback mechanism that can help users adjust their behavior in time to prevent negative outcomes.

7. The system of claim 6 wherein the population data is weighted or upsampled such that data from users of a selected demographic or physiology, that are closer to that of a specific user, are emphasized more strongly when performing BPTCA to discover CCRs and relaying them to the user, further allowing weight higher or upsample the data of the individual user themselves for maximum personalization at the cost of lower effective data volumes.

8. The system of claim 5 wherein a time series vector of multiple interchanging interventions are annotated or obtained through IOT for the user and considered in the BPTCA approach (alongside estimates on how friction full each intervention is), instead of evaluating one intervention at a time, in order to identify a subset of interventions that have a large synergistic impact, while minimizing the required friction to implement them.

9. The system of claim 2 wherein biological age changes predicted from periodic epigenome or other applicable HBD samples, are related to changes in the wearable-ome derived biological age, wherein these QCH are of the same nature (biological age accelerating or decelerating), changes to wearable-ome features are used to communicate potential causes behind these QCH, firstly at the level of physiological features in the wearable-ome (for example a change in pulse waveform health) and secondly at the level of behavioral features of the wearable-ome (e.g. increased vigorous exercise duration).

10. The system of claim 9, wherein FSCC of a subset of UPLGs, are communicated to a user alongside changes in BioAge as additional context on the impact of their efforts to increase longevity and biological age deceleration.

11. A method comprising:
a. collecting raw sensor data from a wearable device;
b. performing digital signal processing of the raw wearable data using a series of algorithms;
c. extracting wearable-derived physiological biometrics (WDPB) from the results of the digital signal processing algorithms;
d. extracting wearable-derived behavioral biometrics (WDBB) from the results of the digital signal processing algorithms;
e. predicting biological age and biological age acceleration using WBAPM trained on physiological and behavioral biometrics as input, based on the output models that predict biological age and biological age acceleration from high-throughput biology data (HBD) through application of the BiologicalAge Fit (BAF) principle; and
f. providing a user interface, exemplified by a wrist-worn wearable, a mobile device, or a laptop/desktop display, to display results.

12. The method of claim 11, further comprising averaging predictions of biological age over several days to reduce day-to-day variability in BiologicalAge Fit (BAF) based models with low-frequency sampling, facilitating trend analysis on a shorter timescale.

13. The method of claim 12, further comprising providing daily predictions to offer more frequent estimates of biological age from wearable data compared to repeated sample collection and HBD production for BAF-based biological age prediction.

14. The method of claim 12, further comprising recording additional behavioral biometrics (ABB) and physiological biometrics (APB) from any other applicable IoT device capable of transmitting lifestyle behavioral information.

15. The method of claim 14, further comprising using a Behavior-Physiology Temporal Causal Analysis (BPTCA) approach to discover statistically significant relationships between lifestyle QCB and subsequent changes in biological age as predicted from wearable device data, with a time delay ranging from one day to one month.

16. The method of claim 15, further comprising Discovering causal relationships between voluntary behavior and wearable-derived biological age on the data of one user at a time, through the aggregation of volumes of longitudinal data on a single user, and sharing personalized insights with said user.

17. The method of claim 15, further comprising discovering candidate causal relationships between voluntary behavior and wearable-derived biological age on the data of a population of users, and extrapolating insights to the behavior recorded for a single user, to communicate the projected consequences of a user's behavior and provide a feedback mechanism for behavior adjustment.

18. The method of claim 15, further comprising annotating or obtaining a time series vector of multiple interchanging interventions through IoT for the user and considering them in the BPTCA approach to identify a subset of interventions with large synergistic impact while minimizing required friction to implement them.

19. The method of claim 12, further comprising relating biological age changes predicted from periodic epigenome samples to changes in wearable-ome derived biological age and communicating potential causes behind these QCH at the level of the physiological and behavioral features of the wearable-ome that comprise them.

20. The method of claim 19, further comprising communicating the functional state of a subset of Universal ProLongevity Genes (UPLGs) alongside changes in BioAge as additional context on the impact of efforts to increase longevity and biological age deceleration.

21. The system of claim 1, wherein the WBAPM for a particular user is continuously personalized by calibrating it through periodic WBD sample collection and determining the difference between WDBAA and EBAA and offsetting that difference in the output of the personalized WBAPM.
